(19) United States
(12) Patent Application Publication
(10) Pub. No.: US 2003/0032157 A1
(43) Pub. Date: Feb. 13, 2003

(54) Polypeptides interactive with Bcl-XL

(76) Inventors: Philip W. Hammond, Ayer, MA (US); Martin C. Wright, Cambridge, MA (US); Julia Alpin, Kirksville, MO (US); Peter Fekkes, Somerville, MA (US)

Correspondence Address: CLARK & ELBING LLP, 101 FEDERAL STREET, BOSTON, MA, 02110, US

(21) Appl. No.: 10/092,750

(22) Filed: Mar. 7, 2002

Related U.S. Application Data
(60) Provisional application No. 60/274,526, filed on Mar. 8, 2001.

(51) Int. Cl. C12N 9/99; C07H 21/04; C12P 21/02; C12N 5/06

(52) U.S. Cl. 435/184; 435/69.2; 435/320.1; 435/325; 536/23.2

(57) ABSTRACT

Described herein are methods and reagents for identifying polypeptides that bind to a Bcl-X_{L} polypeptide, and methods for identifying compounds that modulate the interaction between a Bcl-X_{L}-binding polypeptide and a Bcl-X_{L} polypeptide.

This application claims the benefit of the filing date of United States provisional application, U.S. Ser. No. 60/274,526, filed Mar. 8, 2001, hereby incorporated by reference.

### BACKGROUND OF THE INVENTION

In general, the present invention relates to polypeptides that bind to Bcl-X_{L}, methods for identifying such polypeptides, and methods for identifying compounds that modulate the interaction between a Bcl-X_{L}-binding polypeptide and Bcl-X_{L} .

With the impending completion of the human genome sequence, interest is shifting to the emergent field of proteomics. One critical aspect of proteomics is the creation of a comprehensive map of protein-protein interactions. Such interactions are responsible for most signal transduction, making them attractive targets for drug therapy.

The primary methodology currently in use for interaction mapping is the yeast two-hybrid assay. Recently, genome-wide efforts to map protein-protein interactions have been reported for S. cerevisiae and, to a more limited extent, for C. elegans (Ito et al., Proc. Natl. Acad. Sci. U.S.A. 97:1143-1147, 2000; Uetz et al., Nature 403:623-627, 2000; and Walhout et al., Science 287:116-122, 2000). In the two-hybrid assay, the interaction of two proteins brings together their respective fusion partners, the DNA binding and activation domains of a transcription factor such as GAL4. This interaction thereby increases the transcription of a reporter gene that provides for the identification of interacting pairs.

While the yeast two-hybrid system has emerged as the leading technology in the field of protein-protein interactions, it is not without significant limitations. Firstly, the yeast two-hybrid system is limited by the in vivo nature of the assay. Binding interactions must take place under the conditions in the nucleus of the yeast cell, and many extracellular proteins are unstable under these reducing conditions. In addition, proteins may prove toxic to the yeast through interactions with host cell proteins. Secondly, in order to generate a signal the two protein partners must be fused in an orientation that allows productive binding. Thirdly, because the two-hybrid system is a screening technique, there are practical limitations on the number of colonies that can be assayed.

Display technologies provide a powerful alternative and bypass many of the limitations of the two-hybrid system (Zozulya et al., Nat. Biotechnol. 17:1193-1198, 1999). In display methods, the interaction between a library member and a target polypeptide occurs in vitro, allowing optimal binding conditions to be used for different targets. Additionally, large libraries are screened iteratively, thus allowing even very low copy number proteins to be identified. However, in its most widely practiced form, phage display, this approach has similarly been hampered by the limitations of living systems. Specifically, libraries must be cloned, which decreases representation of the library members, can lead to the loss of sequences unstable in E. coli, and requires that proteins be properly processed to allow assembly of phage particles. In addition, the generation of libraries large enough to cover the entire proteome is difficult.

### SUMMARY OF THE INVENTION

The present invention features the application of mRNA display to the identification of protein-protein interactions involving the anti-apoptotic protein Bcl-X_{L}. The anti-apoptotic activity of Bcl-X_{L} is antagonized through binding to pro-apoptotic members of the Bcl-2 family, and protein members of the Bcl-2 family have been proposed as targets for drug therapy (Kinscherf et al., Expert. Opin. Investig. Drugs 9:747-764, 2000; Mattson and Culmsee, Cell Tissue Res. 301:173-187, 2000; and Chaudhary et al., Environ. Health Perspect. 107 Suppl 1:49-57, 1999). Methods for identifying Bcl-X_{L}-binding polypeptides through mRNA display, as well as polypeptides identified as Bcl-X_{L}-binding polypeptides and the nucleic acid sequences encoding such polypeptides are described herein.

Accordingly, in a first aspect, the invention features a substantially pure human Bcl-X_{L}-binding polypeptide consisting of the sequence of any of SEQ ID NOS: 4-50, 63-71, and 224-228, or containing the sequence of any of SEQ ID NOS: 51-62, 229, and 230, as well as isolated nucleic acid molecules encoding those polypeptides (that is, SEQ ID NOS: 4-71 and 224-230), and vectors and cells containing those isolated nucleic acid molecules. In one embodiment, the nucleic acid molecule consists of the sequence of any of SEQ ID NOS: 156-202, 215-223, and 231-235. In another embodiment, the nucleic acid molecule contains the sequence of any of SEQ ID NOS: 203-214, 236, and 237. In another embodiment, the cell contains the vector into which an isolated nucleic acid molecule encoding a polypeptide of any of SEQ ID NOS: 4-71 and 224-230 is incorporated.

In a second aspect, the invention features a method of identifying a Bcl-X_{L}-binding polypeptide. The method involves providing a population of source labeled nucleic acid-protein fusion molecules; contacting the population of nucleic acid-protein fusion molecules with a Bcl-X_{L} polypeptide under conditions that allow interaction between the protein portion of a nucleic acid-protein fusion molecule of the population and the Bcl-X_{L} polypeptide; and detecting an interaction between the protein portion and the Bcl-X_{L} polypeptide, thereby identifying a Bcl-X_{L}-binding polypeptide. In a preferred embodiment, the population of source labeled nucleic acid-protein fusion molecules is derived from more than one source. In another preferred embodiment, the nucleic acid-protein fusion molecules are detectably-labeled. In yet another preferred embodiment, the Bcl-X_{L} polypeptide is immobilized on a solid support, and the detection of an interaction between the protein portion of a nucleic acid-protein fusion molecule and a Bcl-X_{L} polypeptide is carried out by detecting the labeled nucleic acid-protein fusion molecule bound to the solid support. In this case, the support is preferably a bead or a chip.

In a third aspect, the invention features a method of identifying a compound that modulates binding between a Bcl-X_{L} polypeptide and a Bcl-X_{L}-binding polypeptide. The method entails contacting a Bcl-X_{L} polypeptide with (i) a Bcl-X_{L}-binding polypeptide consisting of the sequence of any of SEQ ID NOS: 4-50, 63-71, and 224-228, or containing the sequence of any one of SEQ ID NOS: 51-62, 229, and 230, and (ii) a candidate compound, under conditions that allow binding between the Bcl-X_{L} polypeptide and the Bcl-X_{L}-binding polypeptide. The level of binding between the Bcl-X_{L} polypeptide and the Bcl-X_{L}-binding polypeptide is then determined. An increase or decrease in the level of binding between the Bcl-X_{L} polypeptide and the Bcl-X_{L}-binding polypeptide, relative to the level of binding between the Bcl-X_{L} polypeptide and the Bcl-X_{L}-binding polypeptide in the absence of the candidate compound, indicates a compound that modulates the interaction between a Bcl-X_{L} polypeptide and a Bcl-X_{L}-binding polypeptide. The modulation may be an increase or a decrease in binding between the Bcl-X_{L} polypeptide and the Bcl-X_{L}-binding polypeptide.

In one embodiment of this aspect of the invention, the Bcl-X_{L}-binding polypeptide is part of a nucleic acid-protein fusion molecule. In a preferred embodiment, the Bcl-X_{L}-binding polypeptide is a free polypeptide that is not part of a fusion. In another preferred embodiment, the Bcl-X_{L} polypeptide is attached to a solid support. In yet another preferred embodiment, the Bcl-X_{L}-binding polypeptide is detectably-labeled, and the level of binding between the Bcl-X_{L} polypeptide and the Bcl-X_{L}-binding polypeptide is determined by measuring the amount of Bcl-X_{L}-binding protein that binds to the solid support. Preferably, the solid support is a chip or a bead.

In a fourth aspect, the invention features a method of source-labeling a nucleic acid-protein fusion molecule. This method involves providing an RNA molecule; generating a first cDNA strand using the RNA molecule as a template; generating a second cDNA strand complementary to the first cDNA strand, the second cDNA strand further including a nucleic acid sequence that identifies the source of the RNA molecule; generating a source labeled RNA molecule from the double stranded cDNA molecule of the previous step; attaching a peptide acceptor to the source labeled RNA molecule generated in the previous step; and in vitro translating the RNA molecule to generate a source labeled nucleic acid-protein fusion molecule.

In a related aspect, the invention features a source-labeled nucleic acid-protein fusion molecule, where the nucleic acid portion of the fusion molecule contains a coding sequence for the protein and a label that identifies the source of the nucleic acid portion of the fusion molecule.

In another related aspect, the invention features a method of identifying the source of the nucleic acid portion of a nucleic acid-protein fusion molecule. The method includes providing a population of nucleic acid-protein fusion molecules, each molecule containing a source label that identifies the source of the nucleic acid portion of the fusion; and determining the identity of the source label, thereby identifying the source of the nucleic acid portion of a nucleic acid-protein fusion molecule. In preferred embodiments, the source label is cell type-specific, tissue-specific, or species-specific. In another preferred embodiment, the population of nucleic acid-protein fusion molecules contains subpopulations of nucleic acid-protein fusion molecules from a plurality of sources.

In any of the above aspects of the invention, the nucleic acid-protein fusion molecule is preferably an RNA-protein fusion molecule, for example, as described by Roberts and Szostak (Proc. Natl. Acad. Sci. U.S.A. 94:12297-302, 1997) and Szostak et al. (WO 98/31700; and U.S. Ser. No. 09/247,190), hereby incorporated by reference. Alternatively, the nucleic acid-protein fusion molecule is a DNA-protein fusion molecule, for example a cDNA-protein fusion molecule. Such molecules are described, for example, in U.S. Ser. No. 09/453,190 and WO 00/32823, hereby incorporated by reference.

By “nucleic acid-protein fusion molecule” is meant a nucleic acid molecule covalently bound to a protein. The nucleic acid molecule may be an RNA or DNA molecule, or may include RNA or DNA analogs at one or more positions in the sequence. The “protein” portion of the fusion is composed of two or more naturally occurring or modified amino acids joined by one or more peptide bonds. “Protein,” “peptide,” and “polypeptide” are used interchangeably herein.

By “substantially pure polypeptide” or “substantially pure and isolated polypeptide” is meant a polypeptide (or a fragment thereof) that has been separated from components that naturally accompany it. Typically, the polypeptide is substantially pure when it is at least 60%, by weight, free from the proteins and naturally-occurring organic molecules with which it is naturally associated. Preferably, the polypeptide is a Bcl-X_{L}-binding polypeptide that is at least 75%, more preferably, at least 90%, and most preferably, at least 99%, by weight, pure. A substantially pure Bcl-X_{L}-binding polypeptide may be obtained, for example, by extraction from a natural source (e.g., a cell), by expression of a recombinant nucleic acid encoding a Bcl-X_{L}-binding polypeptide, or by chemically synthesizing the polypeptide. Purity can be measured by any appropriate method, e.g., by column chromatography, polyacrylamide gel electrophoresis, or HPLC analysis.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides not only include those derived from eukaryotic organisms but also those synthesized in E. coli or other prokaryotes.

By a “Bcl-X_{L}-binding polypeptide” is meant a polypeptide that interacts with a Bcl-X_{L} polypeptide or a fragment of a Bcl-X_{L} polypeptide. The interaction of a Bcl-X_{L}-binding polypeptide with a Bcl-X_{L} polypeptide can be detected using binding assays described herein, or any other assay known to one skilled in the art. In addition, a Bcl-X_{L}-binding polypeptide may be contained in the protein portion of a nucleic acid protein fusion molecule.

By a “Bcl-X_{L} polypeptide” is meant a polypeptide that is substantially identical to the polypeptide sequence of GenBank Accession Number: Z23115, or a fragment thereof. For example, a Bcl-X_{L} polypeptide may consist of amino acids 1 to 211 of GenBank Accession Number: Z23115.

By “substantially identical” is meant a nucleic acid molecule exhibiting at least 50%, preferably, 60%, more preferably, 70%, still more preferably, 80%, and most preferably, 90% identity to a reference nucleic acid sequence or polypeptide. For comparison of nucleic acid molecules, the length of sequences for comparison will generally be at least 30 nucleotides, preferably, at least 50 nucleotides, more preferably, at least 60 nucleotides, and most preferably, the full length nucleic acid molecule. For comparison of polypeptides, the length of sequences for comparison will generally be at least 10 amino acids, preferably, at least 15 nucleotides, more preferably, at least 20 amino acids, and most preferably, the full length polypeptide.

The “percent identity” of two nucleic acid or polypeptide sequences can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, Academic Press, 1987; and Sequence Analysis Primer, Gribskov, and Devereux, eds., M. Stockton Press, New York, 1991; and Carillo and Lipman, SIAM J. Applied Math. 48: 1073, 1988.

Methods to determine identity are available in publicly available computer programs. Computer program methods to determine identity between two sequences include, but are not limited to, the GCG program package (Devereux et al., Nucleic Acids Research 12(1): 387, 1984), BLASTP, BLASTN, and FASTA (Altschul et al., J. Mol. Biol. 215: 403 (1990). The well known Smith Waterman algorithm may also be used to determine identity. The BLAST program is publicly available from NCBI and other sources (BLAST Manual, Altschul, et al., NCBI NLM NIH Bethesda, Md. 20894). Searches can be performed in URLs such as the following http://www.ncbi.nlm.nih.gov/BLAST/unfinishedgenome.html; or http://www.tigr.org/cgi-bin/BlastSearch/blast.cgi. These software programs match similar sequences be assigning degrees of homology to various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By a “compound,” “test compound,” or “candidate compound” is meant a chemical molecule, be it naturally-occurring or artificially-derived, and includes, for example, peptides, proteins, synthetic organic molecules, naturally-occurring organic molecules, nucleic acid molecules, and components thereof.

By a “solid support” is meant any solid surface including, without limitation, any chip (for example, silica-based, glass, or gold chip), glass slide, membrane, bead, solid particle (for example, agarose, Sepharose, polystyrene or magnetic bead), column (or column material), test tube, or microtiter dish.

By a “microarray” or “array” is meant a fixed pattern of immobilized objects on a solid surface or membrane. As used herein, the array is made up of polypeptides immobilized on the solid surface or membrane. “Microarray” and “array” are used interchangeably. Preferably, the microarray has a density of between 10 and 1,000 objects/cm².

By “detectably-labeled” is meant any means for marking and identifying the presence of a molecule, e.g., an oligonucleotide probe or primer, a gene or fragment thereof, a cDNA molecule, or an antibody. Methods for detectably-labeling a molecule are well known in the art and include, without limitation, radioactive labeling (e.g., with an isotope such as ³²P or ³⁵S) and nonradioactive labeling (e.g., with a fluorescent label, such as fluorescein, or a chemiluminescent label).

By a “source label” is meant a nucleic acid sequence that is attached to a nucleic acid-protein fusion molecule. The source label identifies the origin of the nucleic acid portion of a nucleic acid-protein fusion molecule. For example, a source label can identify a specific cell type, tissue type, or species from which the nucleic acid portion of a nucleic acid-protein fusion molecule is derived. The source label also permits the selection of nucleic acid-protein fusion molecules from a particular source from a pool of nucleic acid-protein fusion molecules from various sources. For example, a primer or a probe can be designed to detect the source label of nucleic acid-protein fusion molecules from a particular source, thereby allowing amplification or detection by hybridization of those particular fusion molecules. Such a primer or probe can also be designed for use as a handle for purification of a nucleic acid molecule or a nucleic acid-protein fusion molecule.

By “sequence cluster” is meant a group of sequences that form a continuous single sequence when their overlapping sequences are aligned. For example, a cluster sequence can be a set of sequences that each contain sequences in common with the other members of the sequence cluster. Sequence clusters can be formed using, for example, the computer program MacVector.

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8× SSC, 0.2 M Tris-Cl, pH 7.6, 1× Denhardt's solution, 10% dextran sulfate, and 0.1% SDS, at a temperature of 42° C. (these are typical conditions for high stringency Northern or Southern hybridizations). High stringency hybridization is also relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and may be found, for example, in Ausubel et al., Current Protocols in Molecular Biology, John Wiley & Sons, New York, N.Y., 1998, hereby incorporated by reference.

By “transgene” is meant any piece of DNA that is inserted by artifice into a cell, and becomes part of the genome of the organism that develops from that cell. Such a transgene may include a gene that is partly or entirely heterologous (i.e., foreign) to the transgenic organism, or may represent a gene homologous to an endogenous gene of the organism.

By “transgenic” is meant any cell that includes a DNA sequence that is inserted by artifice into a cell and becomes part of the genome of the organism that develops from that cell. As used herein, the transgenic organisms are generally transgenic mammals (e.g., mice, rats, and goats) and the DNA (transgene) is inserted by artifice into the nuclear genome.

By “knockout mutation” is meant an artificially-induced alteration in the nucleic acid sequence (created via recombinant DNA technology or deliberate exposure to a mutagen) that reduces the biological activity of the polypeptide normally encoded therefrom by at least 80% relative to the unmutated gene. The mutation may, without limitation, be an insertion, deletion, frameshift mutation, or a missense mutation. The knockout mutation can be in a cell ex vivo (e.g., a tissue culture cell or a primary cell) or in vivo.

A “knockout animal” is a mammal, preferably, a mouse, containing a knockout mutation as defined above.

By “transformation,” “transfection,” or “transduction” is meant any method for introducing foreign molecules into a cell, e.g., a bacterial, yeast, fungal, algal, plant, insect, or animal cell. Lipofection, DEAE-dextran-mediated transfection, microinjection, protoplast fusion, calcium phosphate precipitation, retroviral delivery, electroporation, and biolistic transformation are just a few of the methods known to those skilled in the art which may be used. In addition, a foreign molecule can be introduced into a cell using a cell penetrating peptide, for example, as described by Fawell et al. (Proc. Natl. Acad. Sci. U.S.A. 91:664-668, 1994) and Lindgren et al. (TIPS 21:99-103, 2000).

By “transformed cell,” “transfected cell,” or “transduced cell,” is meant a cell (or a descendent of a cell) into which a nucleic acid molecule encoding a polypeptide of the invention has been introduced, by means of recombinant nucleic acid techniques.

By “promoter” is meant a minimal sequence sufficient to direct transcription. If desired, constructs of the invention may also include those promoter elements that are sufficient to render promoter-dependent gene expression controllable in a cell type-specific, tissue-specific, or temporal-specific manner, or inducible by external signals or agents; such elements may be located in the 5′ or 3′ or intron sequence regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “sample” is meant a tissue biopsy, cells, blood, serum, urine, stool, or other specimen obtained from a patient or test subject. The sample is analyzed to detect a mutation in a gene encoding a Bcl-X_{L}-binding polypeptide, or expression levels of a gene encoding a Bcl-X_{L}-binding polypeptide, by methods that are known in the art. For example, methods such as sequencing, single-strand conformational polymorphism (SSCP) analysis, or restriction fragment length polymorphism (RFLP) analysis of PCR products derived from a patient sample may be used to detect a mutation in a gene encoding a Bcl-X_{L}-binding polypeptide; ELISA may be used to measure levels of a Bcl-X_{L}-binding polypeptide; and PCR may be used to measure the level of nucleic acids encoding a Bcl-X_{L}-binding polypeptide.

By “apoptosis” is meant cell death characterized by any of the following properties: nuclear condensation, DNA fragmentation, membrane blebbing, or cell shrinkage.

By “modulating” is meant either increasing (“upward modulating”) or decreasing (“downward modulating”) the number of cells that undergo apoptosis in a given cell population. Preferably, the cell population is selected from a group including cancer cells (e.g., ovarian cancer cells, breast cancer cells, pancreatic cancer cells), leukemic cells, lymphoma cells, T cells, neuronal cells, fibroblasts, or any other cell line known to proliferate in a laboratory setting. It will be appreciated that the degree of apoptosis modulation provided by an apoptosis modulating compound in a given assay will vary, but that one skilled in the art can determine the statistically significant change in the level of apoptosis that identifies a compound that increases or decreases apoptosis. Preferably, for downward modulating, apoptosis is decreased by least 20%, more preferably, by at least, 40%, 50%, or 75%, and, most preferably, by at least 90%, relative to a control sample which was not administered an apoptosis downward modulating test compound. Also as used herein, preferably, for upward modulating, apoptosis is increased by at least 1.5-fold to 2-fold, more preferably, by at least 3-fold, and most preferably, by at least 5-fold, relative to a control sample which was not administered an apoptosis upward modulating test compound.

By an “apoptotic disease” is meant a condition in which the apoptotic response is abnormal. This may pertain to a cell or a population of cells that does not undergo cell death under appropriate conditions. For example, normally a cell will die upon exposure to apoptotic-triggering agents, such as chemotherapeutic agents, or ionizing radiation. When, however, a subject has an apoptotic disease, for example, cancer, the cell or a population of cells may not undergo cell death in response to contact with apoptotic-triggering agents. In addition, a subject may have an apoptotic disease when the occurrence of cell death is too low, for example, when the number of proliferating cells exceeds the number of cells undergoing cell death, as occurs in cancer when such cells do not properly differentiate.

An apoptotic disease may also be a condition characterized by the occurrence of inappropriately high levels of apoptosis. For example, certain neurodegenerative diseases, including but not limited to Alzheimer's disease, Huntington's disease, Parkinson's disease, amyotrophic lateral sclerosis, multiple sclerosis, restenosis, stroke, and ischemic brain injury are apoptotic diseases in which neuronal cells undergo undesired cell death.

By “proliferative disease” is meant a disease that is caused by or results in inappropriately high levels of cell division, inappropriately low levels of apoptosis, or both. For example, cancers such as lymphoma, leukemia, melanoma, ovarian cancer, breast cancer, pancreatic cancer, and lung cancer are all examples of proliferative disease.

By a “substantially pure nucleic acid,” “isolated nucleic acid,” or “substantially pure and isolated nucleic acid” is meant nucleic acid (for example, DNA) that is free of the genes which, in the naturally-occurring genome of the organism from which the nucleic acid of the invention is derived, flank the nucleic acid. The term therefore includes, for example, a recombinant DNA that is incorporated into a vector; into an autonomously replicating plasmid or virus; or into the genomic DNA of a prokaryote or eukaryote; or that exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By “antisense,” as used herein in reference to nucleic acids, is meant a nucleic acid sequence, regardless of length, that is complementary to the coding strand of a nucleic acid molecule encoding a Bcl-X_{L} polypeptide or a Bcl-X_{L}-binding polypeptide. Preferably, the antisense nucleic acid molecule is capable of modulating apoptosis when present in a cell. Modulation of at least 10%, relative to a control, is recognized; preferably, the modulation is at least 25%, 50%, or more preferably, 75%, and most preferably, 90% or more.

By a “purified antibody” is meant an antibody that is at least 60%, by weight, free from proteins and naturally occurring organic molecules with which it is naturally associated. Preferably, the preparation is at least 75%, more preferably, 90%, and, most preferably, at least 99%, by weight, antibody, e.g., a Bcl-X_{L}-binding polypeptide-specific antibody. A purified antibody may be obtained, for example, by affinity chromatography using recombinantly-produced protein or conserved motif peptides and standard techniques.

By “specifically binds” is meant a compound that recognizes and binds a protein or polypeptide, for example, a Bcl-X_{L} polypeptide or a Bcl-X_{L}-binding polypeptide, and that when detectably labeled can be competed away for binding to that protein or polypeptide by an excess of compound that is not detectably labeled. A compound that non-specifically binds is not competed away by the above excess detectably labeled compound.

The present invention has several utilities. Since the Bcl-2 family of proteins, and Bcl-X_{L} itself, has been implicated in apoptosis, these Bcl-2 family polypeptides can be used in screens for therapeutics that modulate diseases or developmental abnormalities involving overactivity or underactivity of apoptotic pathways. In particular, Bcl-X_{L} is known to protect cancer cells (e.g., pancreatic carcinoma cells) from stimulation of apoptosis, and this effect is reversible by adding an agent, Bax (Hinz et al., Oncogene 19:5477-5486, 2000), that binds to Bcl-X_{L} at the same site as many of the polypeptides of the present invention. Therefore, the polypeptides that bind to Bcl-X_{L}, described herein, may be used as targets in therapeutics screening assays. The identified polypeptides are particularly useful in such screens because they represent the functional portions of human proteins that bind Bcl-X_{L}. These polypeptides may also be used to detect Bcl-X_{L} polypeptides in a sample. In addition, the methods of the present invention are useful as high-throughput screening methods for potential therapeutics involved in the overactivity or underactivity of apoptotic pathways.

The general approach of the present invention also provides a number of advantages. For example, direct mRNA display allows the mapping of protein-protein interactions, which is useful for drug screening. In mRNA display (Roberts and Szostak, supra), a DNA template is used to transcribe an engineered-mRNA molecule possessing suitable flanking sequences (e.g., a promoter; a functional 5′ UTR to allow ribosome binding; a start codon; an open reading frame; a sequence for polypeptide purification; and a conserved sequence used for ligation to a complementary linker containing puromycin). To the 3′ end of the mRNA, a linker strand with a puromycin moiety (Pu) is then added, preferably by photo-crosslinking. When this RNA is translated in vitro, the puromycin becomes incorporated at the C-terminus of the nascent peptide. The resulting mRNA display construct is then purified after ribosome dissociation. A cDNA strand is then synthesized to protect the RNA and to provide a template for future PCR amplification. A library of such constructs can be incubated with immobilized target, and molecules that bind are enriched by washing away unbound material. Bound cDNAs are recovered, for example, by KOH elution, and subsequent PCR is performed to regenerate a library enriched for target-binding peptides. FIG. 1 shows the steps involved in mRNA display.

As mRNA display is a completely in vitro technique, many of the problems inherent in cloning and expression are eliminated. The elimination of cloning bottlenecks in library preparation allows the generation of very large libraries, routinely in the range of 10¹³ members. In addition, the formation of mRNA display constructs is readily achieved in a mammalian expression system, thereby providing suitable chaperones for the folding of human proteins and the potential for appropriate post-translational modifications.

Other features and advantages of the invention will be apparent from the following detailed description, and from the claims.

### BRIEF DESCRIPTION OF THE DRAWINGS

FIG. 1 is a schematic representation of iterative selection using mRNA display.

FIG. 2A shows the sequences of positive control polypeptides used in Bcl-X_{L} polypeptide binding assays (SEQ ID NOS: 238-240).

FIG. 2B is a graph showing the binding of control polypeptides to a Bcl-X_{L} polypeptide.

FIG. 3A shows polypeptides identified as Bcl-X_{L} binding polypeptides using methods described herein (SEQ ID NOS: 1-71), as well as information on the binding affinity and specificity of the polypeptides. In addition, the number of clones of each sequence cluster obtained from each library is presented.

FIG. 3B shows the polypeptide sequences of Bcl-X_{L}-binding polypeptides (SEQ ID NOS: 1-71), and indicates corresponding nucleic acid sequences.

FIG. 3C shows the nucleotide sequences of selected Bcl-X_{L}-binding polypeptides (SEQ ID NOS: 72-142). The nucleotide sequences encoding the selected Bcl-X_{L}-binding polypeptides are underlined (SEQ ID NOS: 153-223).

FIG. 4 is a schematic representation of the alignment of selected Bcl-X_{L}-binding polypeptides within their parental proteins. Each unique fragment was analyzed to determine the location of the amino- and carboxyl-termini within the parental protein sequence, and these amino acids are indicated by residue and number. The number of isolated clones corresponding to each unique fragment was determined and is indicated next to the fragment ID. These fragments are mapped against the parental sequences of Bim, Bax, HSPC300, and TPR (SEQ ID NOS: 241-244). The BH3 domain core sequence is underlined for the BimL and Bax proteins. Splice variants are indicated by a * in the ID and the use of (−) in place of (=) in the fragment map.

FIG. 5 is a graph of the relative binding affinity of a selected Bak Bcl-X_{L}-binding polypeptide to immobilized Bcl-X_{L} polypeptide versus concentration of immobilized Bcl-X_{L} polypeptide.

FIG. 6 is a graph of the effect of the binding of a Bcl-X_{L}-binding polypeptide in the presence of a competitor BH3 domain from the Bcl-2 family member Bak.

FIG. 7 shows the polypeptide sequences of representative clones (SEQ ID NOS: 1, 5, 245, 60, 61, 6, 46, 2, 33, 4, 7-10, 3, 11, 48, 12, 53, and 54) from sequence clusters that were bound to a Bcl-X_{L} polypeptide in the presence of a competitor BH3 domain from the Bcl-2 family member Bak. Competitive binding was determined relative to a control containing no competitor. The selected polypeptide sequences are shown aligned by sequence homology, where possible, to the known BH3 domains of Bim, Bak, and Bax.

FIGS. 8A, 8B, and 8C are tables of amino acid sequences that bind Bcl-X_{L} protein (FIGS. 8A and 8B; SEQ ID NOS: 224-230) and their nucleic acid coding sequences (FIG. 8C; SEQ ID NOS: 231-237).

FIG. 9 is a graph showing free peptide binding to GST-BCL-X_{L}, as compared to background binding to GST. Bax was used as a positive control for Bcl-X_{L} binding.

Described herein are methods for identifying polypeptides that interact with a Bcl-X_{L} polypeptide; methods for identifying compounds that increase or decrease the binding between a Bcl-X_{L} polypeptide and a Bcl-X_{L}-binding polypeptide; methods for source labeling a nucleic acid-protein fusion molecule; and methods for identifying the source of the nucleic acid portion of a nucleic acid-protein fusion molecule. Techniques for carrying out each method of the invention are now described in detail, using particular examples. The examples are provided for the purpose of illustrating the invention, and should not be construed as limiting. Also described herein are novel Bcl-X_{L}-binding polypeptides and nucleic acid molecules obtained through the methods of the present invention.

Materials and Methods for Identifying Bcl-X_{L}-Binding Polypeptides

The experiments described herein were carried out using the materials and methods described below.

Choice of UTR Sequence Tags

Unique UTR sequences that are compatible with translation in rabbit reticulocyte lysate were identified by selection from a library of c-myc mRNAs with a partially randomized 5′ UTR. The c-myc construct described by Roberts and Szostak (supra) was amplified by PCR using the 5′ primer TAA TAC GAC TCA CTA TAG GGA CAA TTA CTA TTT ACA ATT HHH HHH HHA CAA TGG CTG AAG AAC AGA AAC TG (where H is an equimolar mixture of A, C, and T) (SEQ ID NO: 143). This amplification inserted 8 random bases into the 5′ UTR upstream of the ATG start codon, to give a library of 3⁸ (6561) different mRNA molecules after in vitro transcription with T7 RNA polymerase. Fusion formation, reverse transcription, and immunoprecipitation with an anti-c-myc antibody were carried out as described by Roberts and Szostak (supra) to separate mRNAs that had undergone translation from those that had not. The successfully translated and fused sequences were amplified by PCR using the 5′ primer TAATACGACTCA CTATAGGGACAATTACTATTTACAATT (SEQ ID NO: 144), in which the T7 promoter is underlined, to preserve the information in the randomized region. Sequences obtained from individual clones were subsequently used in the construction of tagged libraries.

Library Preparation

The design of the above-described sequence tags can be adapted to source label nucleic acid sequences from various sources. Instead of each sequence tag being a unique sequence (as described above), one sequence tag (source label) is used to label a set of nucleic acid sequences derived from the same cell, tissue, or species. The source labeled sequences can then be pooled with different source labeled sequences and used for mRNA display as described herein, and the origin of each sequence in the pool can be determined.

Individual RNA sequences are translated in vitro, and RNA-protein fusions are formed, for example, according to the methods of Roberts and Szostak (supra) and Szostak et al. (WO 98/31700; U.S. Ser. No. 09/247,190), hereby incorporated by reference. Specifically, each mRNA display library was prepared according to the following methods. Poly-A+mRNA (Clontech) was primed using the oligonucleotides GGAACTTGCTTCGTCTTTGCAATCN₉ (SEQ ID NO: 145) or GGATGATGCTTCGTCTTTGTAATCN₉ (SEQ ID NO: 146) and a cDNA molecule was synthesized using SuperScript II Reverse Transcriptase (Promega). Two primers were used initially, to allow the investigation of different ligation sequences; these sequences were subsequently altered and made uniform by the use of a single PCR primer under conditions that would allow it to anneal to either template. The RNA/cDNA hybrid molecule was then treated with RNase H in order to partially degrade the RNA member of the hybrid molecule. Unextended primers were then removed by purification over an S-300 (Pharmacia) size exclusion column.

Second strand cDNA synthesis was carried out by the Klenow fragment of E. coli DNA polymerase, using primers having the sequence GGACAATTACTATTTACAATT[H₈]ACAATGN₉ (SEQ ID NO: 147) that included a 5′ UTR with a sequence tag H₈ (source label), derived as described above, and a start codon (underlined). In the production of libraries from human kidney, liver, bone marrow, and brain mRNAs, the source labels CTCCTAAC (SEQ ID NO: 250), CTTTCTCT (SEQ ID NO: 251), CTTACTTC (SEQ ID NO: 252), and ATTTCAAT (SEQ ID NO: 253) were used, respectively. Unextended primers were again removed by S-300 size exclusion chromatography, and the cDNA product was then PCR amplified using a forward primer encoding the T7 promoter (underlined) and 5′ UTR, TAATACGACTCACTATAGGGACAATTACTATTTACAATT (SEQ ID NO: 148), and reverse primers corresponding to the fixed regions of the first strand primers above. After PCR product purification using spin columns (Qiagen), short fragments were removed by S-300 size exclusion chromatography.

mRNA Display Construct Formation

The above described PCR products were reamplified using the forward primer described above (TAATACGACTCACTATAGGGACAATTACTATTTACAATT) (SEQ ID NO: 148) and a single reverse primer, TTTTAAATAGCGCATGCCTTATCGTCATCGTCTTTGTAATC (SEQ ID NO: 149), encoding the FLAG-M2 epitope (underlined) and a region complementary to the photoligation linker (italics). The single reverse primer was used to amplify libraries containing each of the initial first strand primer sequences in order to produce a single uniform end. These amplicons were then used as templates for transcription using T7 RNA polymerase (Ambion MegaScript). The resulting RNA molecules were purified by phenol/chloroform/isoamyl alcohol extraction and NAP column (Pharmacia) purification. The puromycin-containing linker 5′-Pso-TAGCGGATGCA₁₈XXCCPu (where X is PEG spacer 9; Pso is psoralen; and Pu is puromycin) was photo-ligated to the 3′ end of the RNA essentially as described by Kurz et al. (Nucleic Acids Res. 28:E83, 2000). Ligated RNA molecules were then translated for 30 min at 30° C. in a 300 μl reaction containing 200 μl of rabbit reticulocyte lysate (Ambion), 120 pmole of ligated RNA, 10 μl of an amino acid mix lacking methionine (Ambion), and 15 μl of ³⁵S-met (Amersham). Subsequently, 100 μl of 2M KCl and 25 μl of 1 M MgCl₂ was added to facilitate formation of the mRNA display complex. The mRNA display constructs were then purified by binding to 100 μl of 50% oligo-dT cellulose slurry in a total volume of 10 ml (100 mM Tris-HCl (pH 8), 10 mM EDTA, 1M NaCl, and 0.25% Triton X-100) at 4° C. for 1 hr. The binding reaction was then transferred to a column (BIORAD), washed 3 times with 1 ml of binding buffer containing no EDTA, then eluted with 100 μl aliquots of 2 mM Tris-HCl (pH 8), 0.05% Triton X-100, and 5 mg/ml BSA.

A cDNA strand was synthesized using SuperScript II RT (Promega) and the reverse sense PCR primer in the manufacturer supplied buffer. The reverse transcription reaction was then diluted to 1 ml in TBK buffer (50 mM Tris-HCl (pH 7.5), 150 mM KCl, and 0.05% Triton X-100) and incubated with 200 μl of anti-FLAG Ab immobilized on agarose beads (Sigma) for 1 hr at 4° C. The binding reaction was transferred to a column and the beads were washed 3 times with 1 ml of TBK buffer. mRNA-display constructs were then eluted with 100 μl aliquots of TBK buffer containing 100 μM FLAG-M2 peptide, 5 mg/ml BSA, and 0.1 mg/ml salmon sperm DNA. The yield of mRNA-protein fusion product was determined by scintillation counting the purified product and comparing it to an estimated specific activity of methionine based on an approximate concentration of 51M in the lysate. For the libraries containing a heterogeneous population of proteins, the prevalence of methionine was approximated as one initiator methionine per molecule plus one for each 60 amino acids.

Target Protein Preparation

A portion of the human Bcl-X_{L} gene was PCR amplified from a GeneStorm® Expression-Ready Bcl-X_{L} clone (Invitrogen, Carlsbad, Calif.) using the primers AGTATCGAATTCATGTCTCAGAGCAACCGG (SEQ ID NO: 150) and TACAGTCTCGAGCTAGTTGAAGCGTTCCTGGCCCT (SEQ ID NO: 151). The 644 nucleotide Bcl-X_{L} DNA fragment obtained from the above PCR reaction was then cloned into the expression vector 4T-1 (Pharmacia). Competent E. coli (BL21 (DE3) pLysS) were transformed with the expression vector and grown on LB/carbenicillin plates overnight at 37° C. A single transformed colony was then selected and grown overnight in 5 ml of LB/carbenicillin. Two ml of this starter culture was used to inoculate a fresh 100 ml culture, which was grown at 37° C. until an OD₆₀₀ of 0.6 was reached.

Expression of the Bcl-X_{L} polypeptide was induced in the bacterial culture by the addition of IPTG to a final concentration of 0.4 mM, and the culture was shaken at 25° C. overnight. The bacterial cells were then harvested for their Bcl-X_{L} polypeptide by centrifugation at 12,000 g for 30 minutes. The cell pellet was resuspended in 1/10 volume 100 mM Tris/HCl (pH 8.0)/100 mM NaCl/0.1% Triton X-100/1.0% glycerol, and the cells were lysed by dounce homogenization and three freeze/thaw cycles. The bacterial cell lysate was clarified by centrifugation at 16,000 g for 30 minutes, and 5 ml of the clarified lysate was applied to a 2 ml RediPack glutathione column (Pharmacia). The column was washed with 20 ml of lysis buffer and eluted, in a stepwise manner, with lysis buffer to which reduced glutathione had been added, to final concentrations of 1, 5, 10, 15, and 20 mM. Fractions of the eluate were analyzed on 4-12% NuPAGE gels (Novex) and positive fractions, based on polypeptide size, were pooled. The protein was dialyzed against 100 mM Tris/HCl (pH 8.0)/100 mM NaCl/0.05% Triton X-100/1.0% glycerol and the protein concentration was determined by BCA assay (Pierce).

Assay to Detect Binding of a Polypeptide to a Bcl-X_{L}-GST Fusion Protein

Detection of a polypeptide binding to a Bcl-X_{L}-glutathione S-transferase (GST) fusion protein was carried out as follows. Twenty microliters of glutathione Sepharose 4B slurry (AP Biotech) was aliquoted to a microcentrifuge tube and washed with PBS. The Bcl-X_{L}-GST fusion protein (60 μg), prepared as describe above, was added and allowed to bind to the Sepharose beads for 1 hr at 4° C. The beads were then re-washed in selection buffer (50 mM Tris-HCL pH 7.5, 150 mM KCl, 0.05% Triton X-100, 0.5 mg/ml BSA, and 0.1 mg/ml salmon sperm DNA). The Bcl-X_{L}-GST beads were resuspended in 100 μl of selection buffer (approximately 11.5 μM Bcl-X_{L}) and ³⁵S-labeled mRNA display construct or free peptide was added (approximately 10-60 nM) and incubated on a rotator for 1 hr at 4° C. The reaction was then transferred to a microcentrifuge column (BioRad) and unbound mRNA display constructs or free peptides were removed by a 10 sec spin at 1,000 rpm. The Sepharose beads were then washed three times with 500 μl of selection buffer. The extent of binding between the Bcl-X_{L}-GST fusion protein and the mRNA display constructs or free peptides was determined by scintillation counting each fraction, including the recovered beads.

Selection

A human Bcl-X_{L}-GST fusion protein was immobilized on Sepharose beads as described above for the binding assay and incubated with the mRNA display library. For the first round of selection, the input was approximately 0.06 pmol of each of the four source labeled libraries from human tissues (kidney, liver, bone marrow, and brain), which were mixed prior to selection. For subsequent rounds of selection, the input of each of the four source labeled libraries ranged from 0.25 to 0.92 pmol in total. After washing the beads of any unbound nucleic acid-protein fusion library members, the cDNA strand of the bound fusions were recovered in three elutions with 100 μl of 0.1N KOH. Eluates were subsequently neutralized by the addition of 2 μl of 1N Tris-HCL pH 7 and 8 μl of 1N HCL. A small scale PCR optimization was performed with the eluate to determine the number of cycles required to produce a strong signal without overamplification (typically 18-28 cycles). The library was then regenerated by PCR using the remainder of the eluate.

Cloning and Sequencing of Library Members that Bind to the Bcl-X_{L}-GST Fusion Protein

PCR products of the selected library members that bound to the Bcl-X_{L}-GST fusion protein were cloned into the TOPO-TA vector (Invitrogen) and, after isolation of individual colonies, the plasmids were purified (Qiagen) and sequenced using standard sequencing techniques (Ausubel et al., supra).

In Vitro Synthesis of Polypeptides that Bind to the Bcl-X_{L}-GST Fusion Protein

To synthesize polypeptides that interacted with the Bcl-X_{L}-GST fusion protein, RNAs were prepared from the PCR products of the selected library members that bound to the Bcl-X_{L}-GST fusion protein and purified as described above. After translation in rabbit reticulocyte lysate (Ambion), the peptides were purified directly from the lysate by immunoprecipitation and peptide elution based on a C-terminal FLAG-M2 epitope contained in the peptide (Sigma).

Detection of Known Bcl-X_{L}-Binding Polypeptides

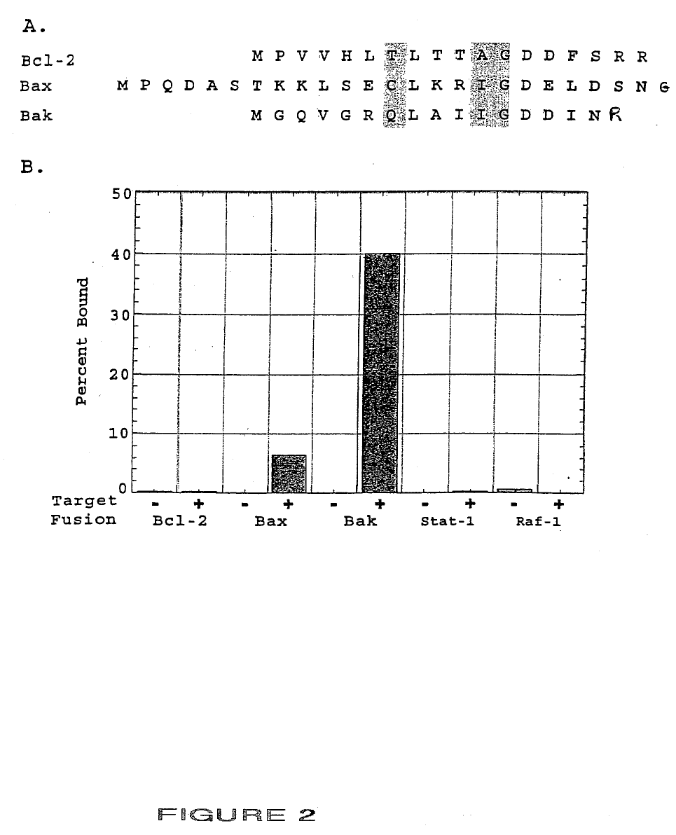

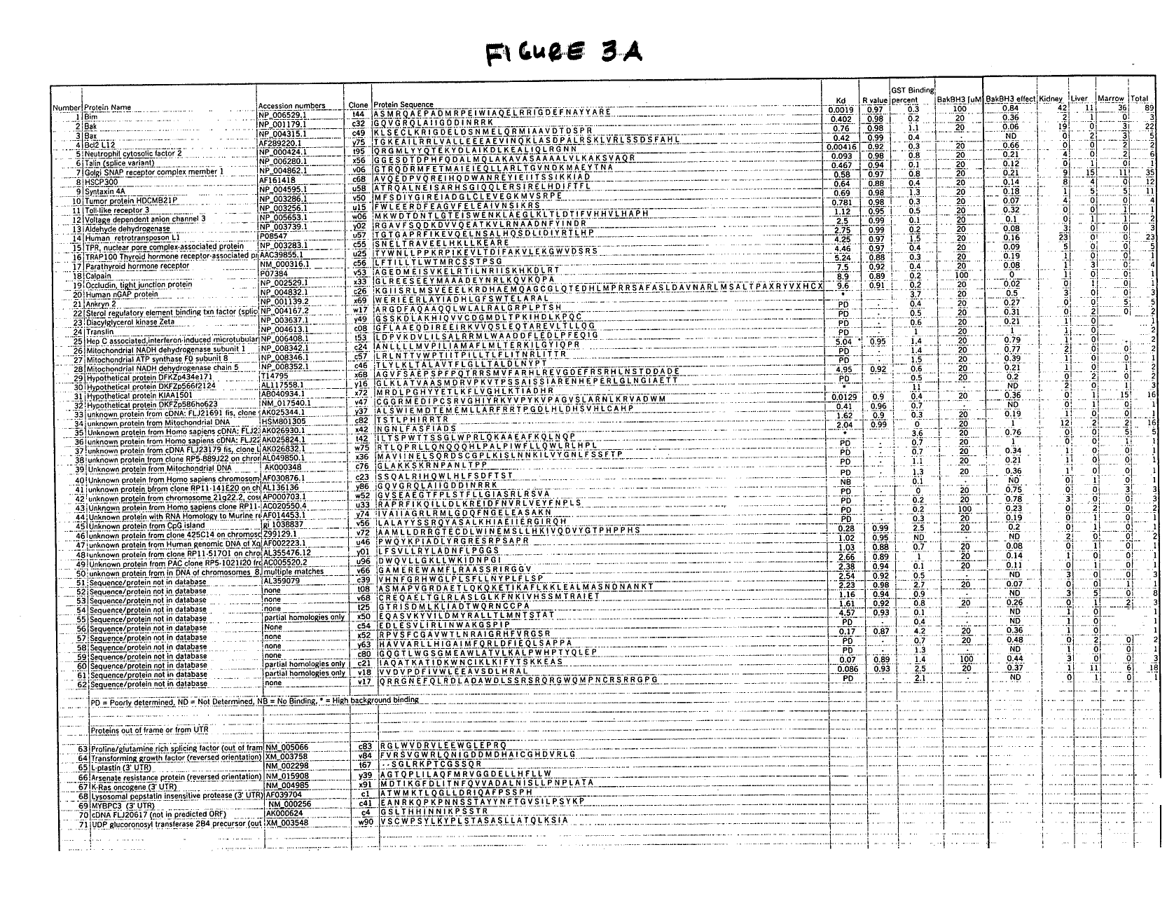

Members of the Bcl-2 family of apoptotic proteins function via homo- and heterodimerization, occurring primarily through the binding of a single α-helix designated the BH3 domain (Bcl-2 Homology domain 3) in a corresponding pocket produced by three α-helices in the interacting partner (Diaz et al., J. Biol. Chem. 272:11350-11355, 1997; and Sattler et al., Science 275:983-986, 1997). The target protein used herein was the human Bcl-X_{L} protein produced as a GST fusion and immobilized on glutathione Sepharose beads. The BH3 domains of three different Bcl-2 family proteins (Bcl-2, Bax, and Bak) were prepared as mRNA display constructs, as described herein, along with control peptides derived from unrelated proteins Stat-1 and Raf-1. The BH3 domains of Bcl-2, Bax, and Bak are shown with the consensus regions aligned and highlighted in FIG. 2A. Individual mRNA display constructs were incubated with either the target Bcl-X_{L}-GST fusion protein bound to glutathione beads or with the beads alone. Unbound materials were collected, and the beads were washed. The amount of peptide bound to the beads was determined by scintillation counting and graphed as the percent of input counts bound (FIG. 2B).

Binding of Bcl-2, Bax, and Bak to Bcl-X_{L}-GST fusion protein was specific to the BH3 helices, with Bak binding most efficiently (40%) followed by Bax (6%); no binding was observed for the BH3 helix from Bcl-2 or either control. The ordering of Bax and Bak is in good agreement with published IC₅₀. values which indicate that Bcl-X_{L} has an affinity for the Bak BH3 domain that is approximately five-fold higher than that for Bax (Diaz et al., supra). The lack of binding observed for the BH3 domain of Bcl-2 could be due to the BH3 domain peptide failing to form a helix (Zhang et al., Biochem. Biophys. Res. Commun. 208:950-956, 1995; and Xie et al., Biochemistry 37:6410-6418, 1998), or the affinity may be below that required to generate a signal in this assay.

Identification of Novel Bcl-X_{L}-Binding Polypeptides

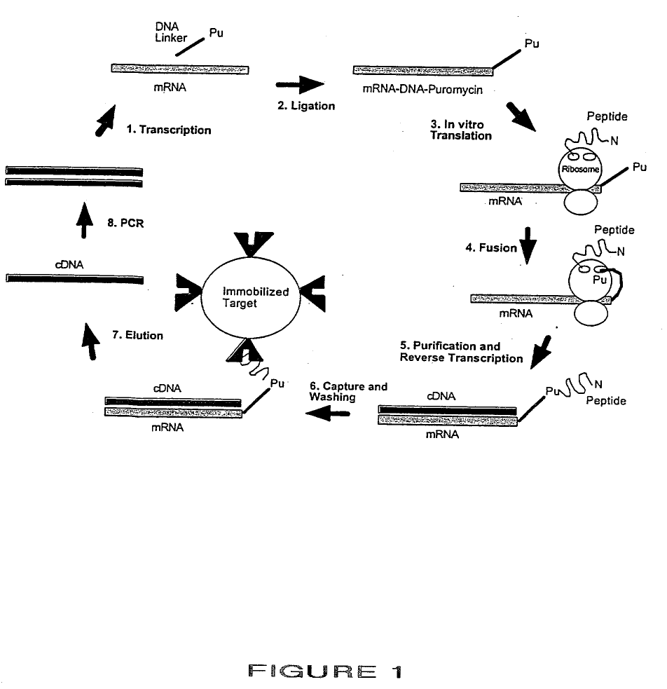

Having established the binding of Bcl-X_{L} control peptides, as described above, a selection to identify binders from within the complex mixture of an mRNA display library was initiated (FIG. 1). Four libraries, individually prepared from the tissue-specific mRNAs of kidney, liver, bone marrow, and brain were pooled prior to initiating selection. Each library contained a unique 8 nucleotide (nt) tag (source tag) within the 5′ UTR to allow specific amplification of an individual library. The ability to mix libraries not only increased the size and diversity of the starting pool, but the identification of tissue of origin for each selected protein provided information similar to that normally obtained from mRNA expression analysis.

As a target for the selection, a GST fusion protein of Bcl-X_{L} was immobilized on glutathione Sepharose beads. The selection was initiated with a combined library of approximately 1.5×10¹¹ molecules. After incubation of the library with the target, unbound members of the library were washed away and the bound material was eluted. An enriched library was then regenerated by PCR, transcription, ligation, translation, fusion, reverse-transcription, and purification. This enriched library was then used for the subsequent round of selection.

After four rounds of selection, the enriched pool from the combined libraries bound the Bcl-X_{L} target at about 40%, an extent similar to the Bak control construct (see FIG. 2B). In order to determine if the selected Bcl-X_{L} polypeptides originated from one or multiple libraries, each library was prepared individually after specific amplification using library specific primers. The library constructed from brain mRNA was omitted due to cross-reaction of the PCR amplification primer. A test of binding revealed that each tissue-specific library bound to the target to an extent similar to the mixed pool. The bound material from each of the individual libraries was then recovered by elution, PCR amplified, and analyzed by cloning and sequencing.

Additional rounds of selection may change the population distribution significantly. A rare sequence from the starting pool that binds tightly might be enriched only to the point of appearing once among the clones while a poorer binding sequence that was abundant in the starting pool might still be found at high copy number. Also, sequencing more clones may lead to the identification of other proteins still present at low copy number.

Sequence Analysis of Bcl-X_{L} Binding Polypeptides

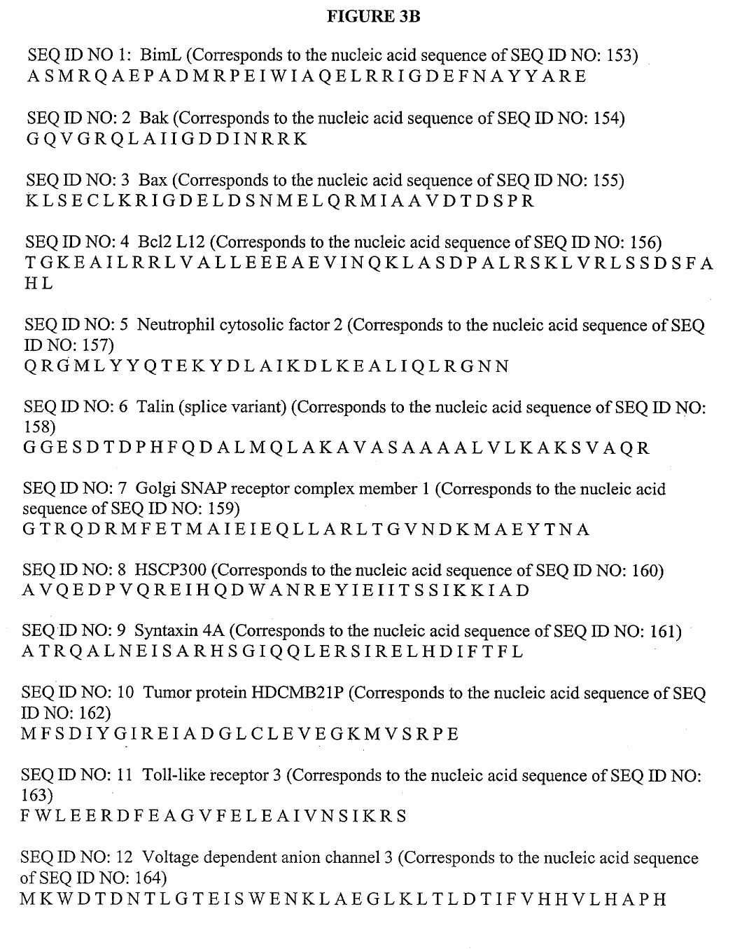

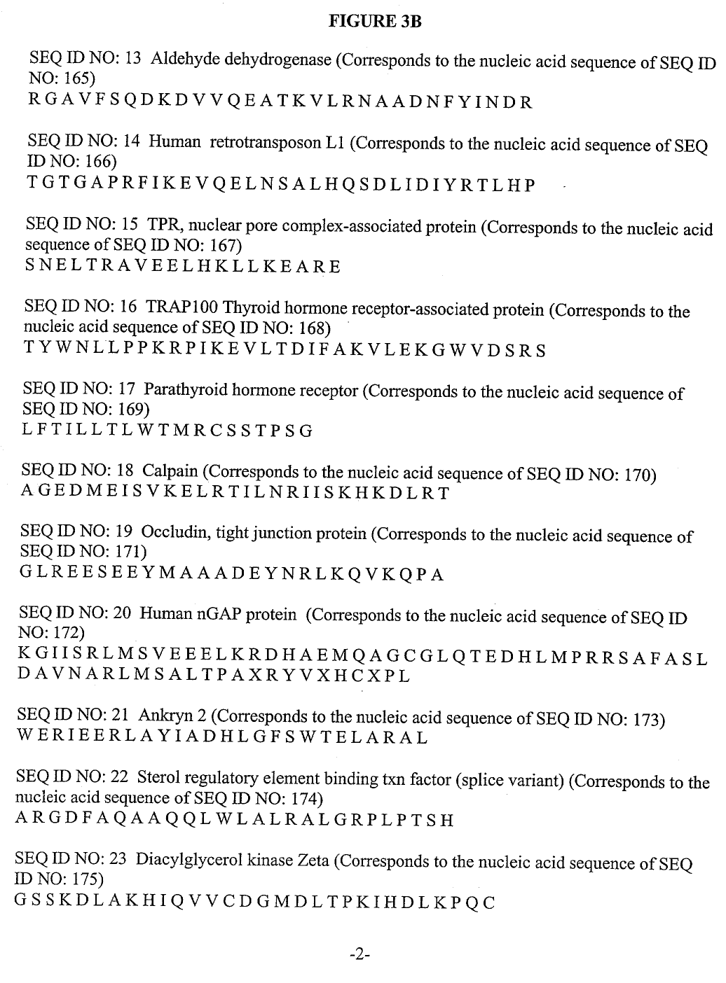

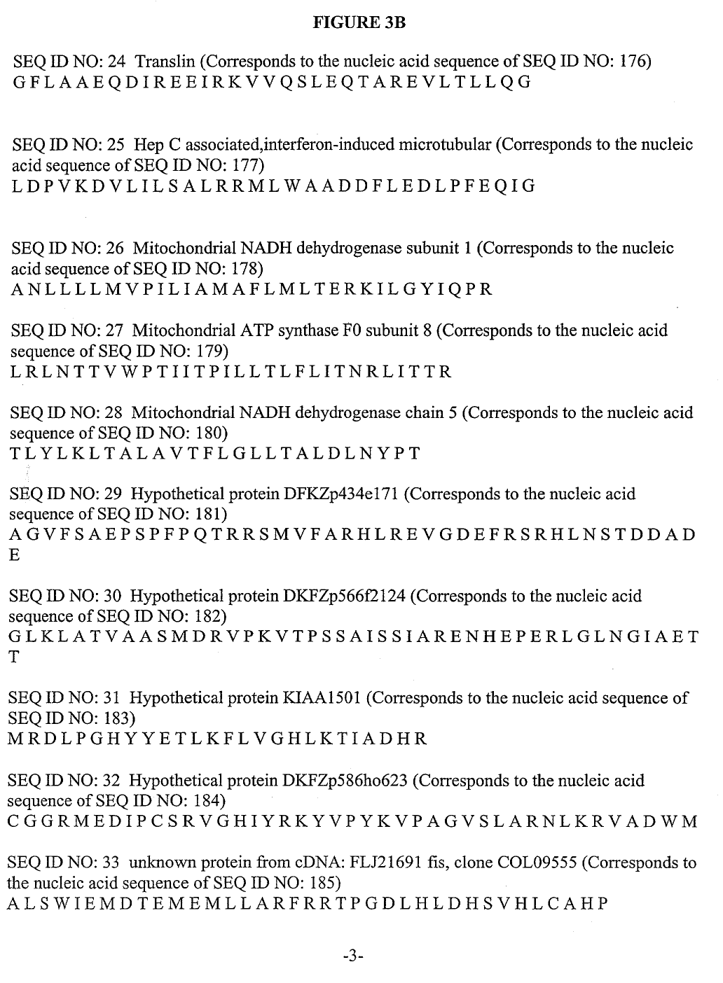

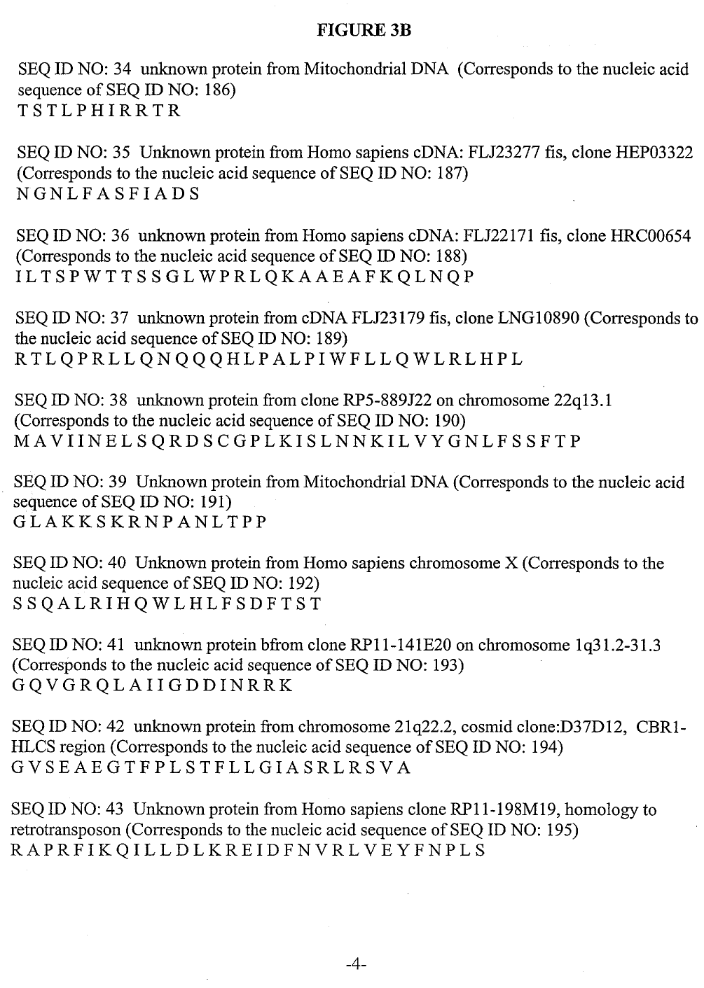

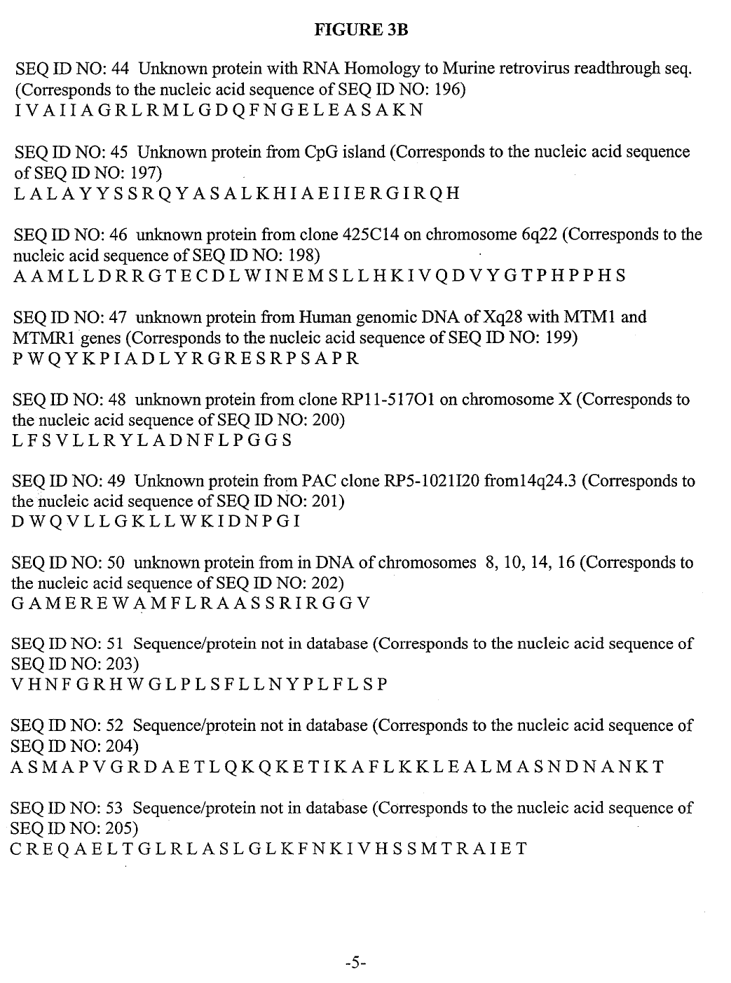

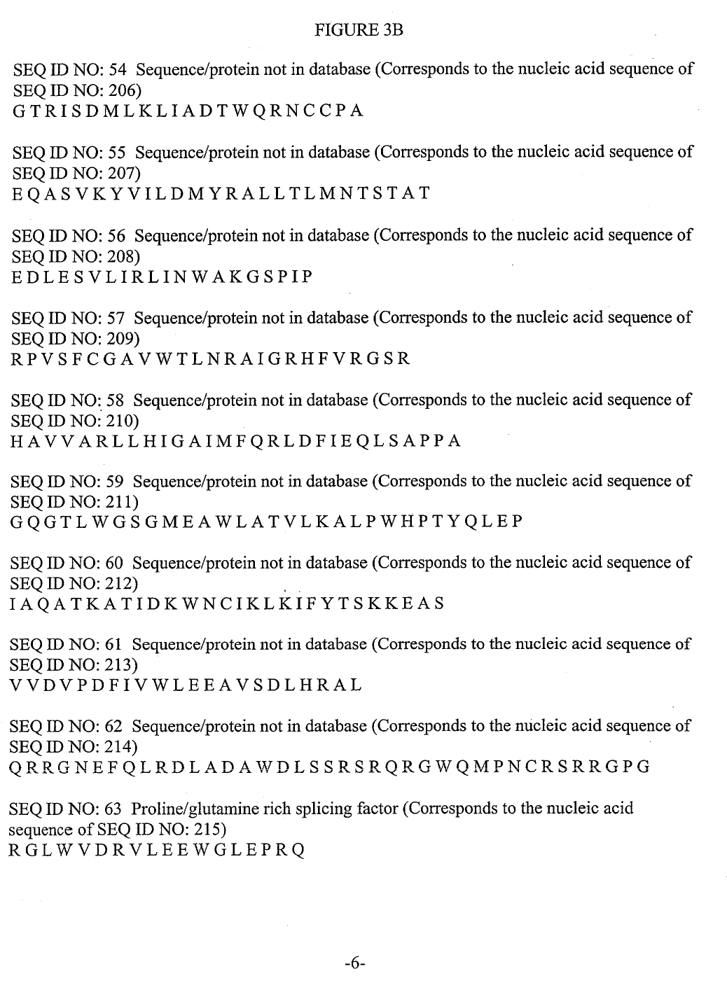

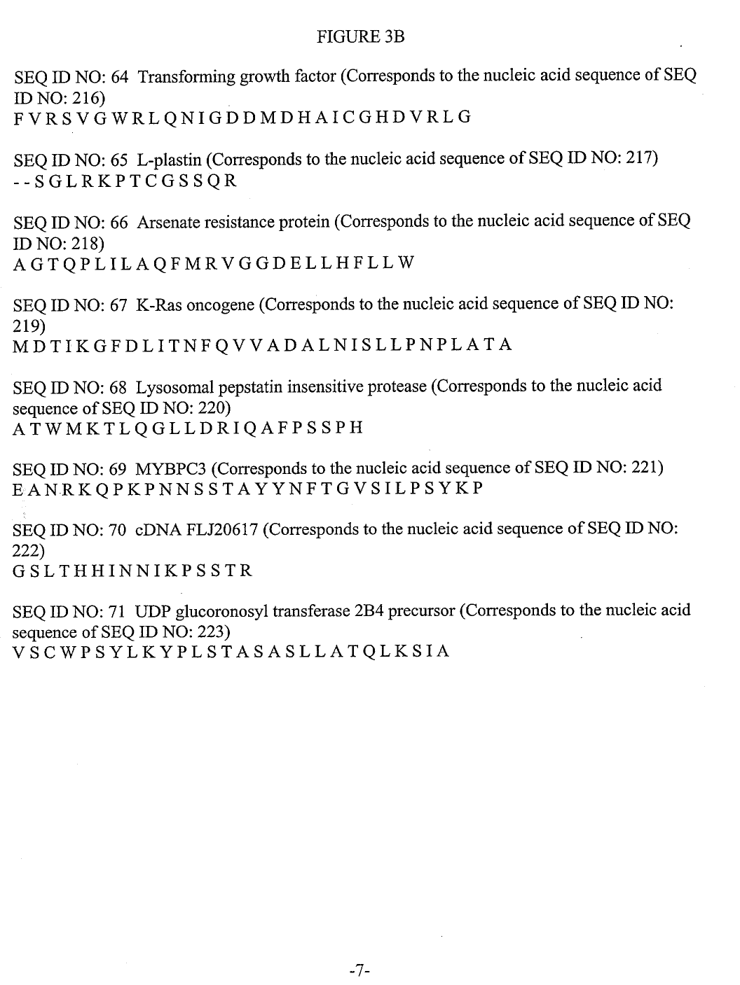

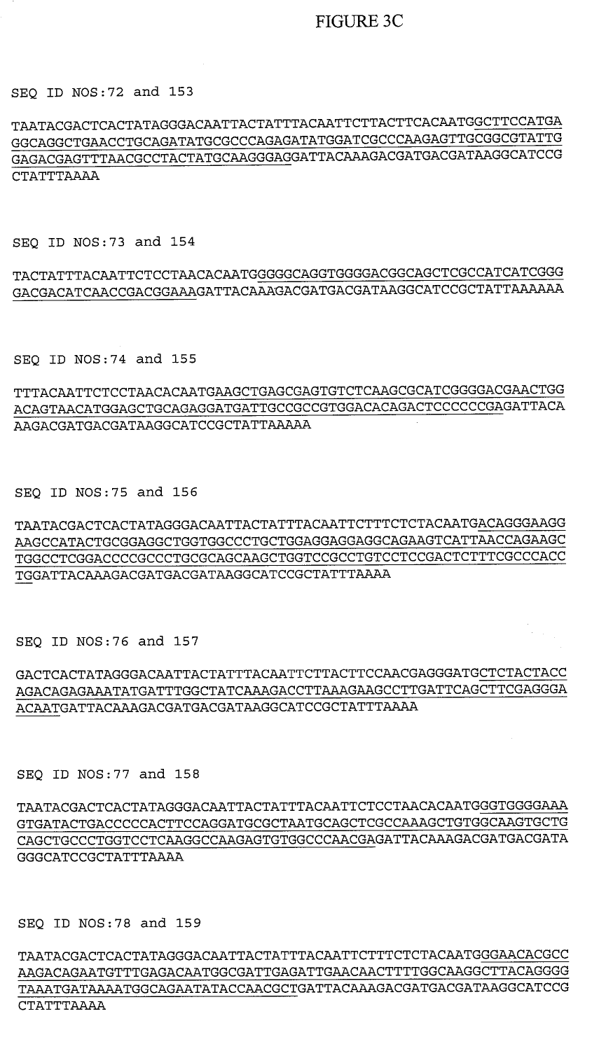

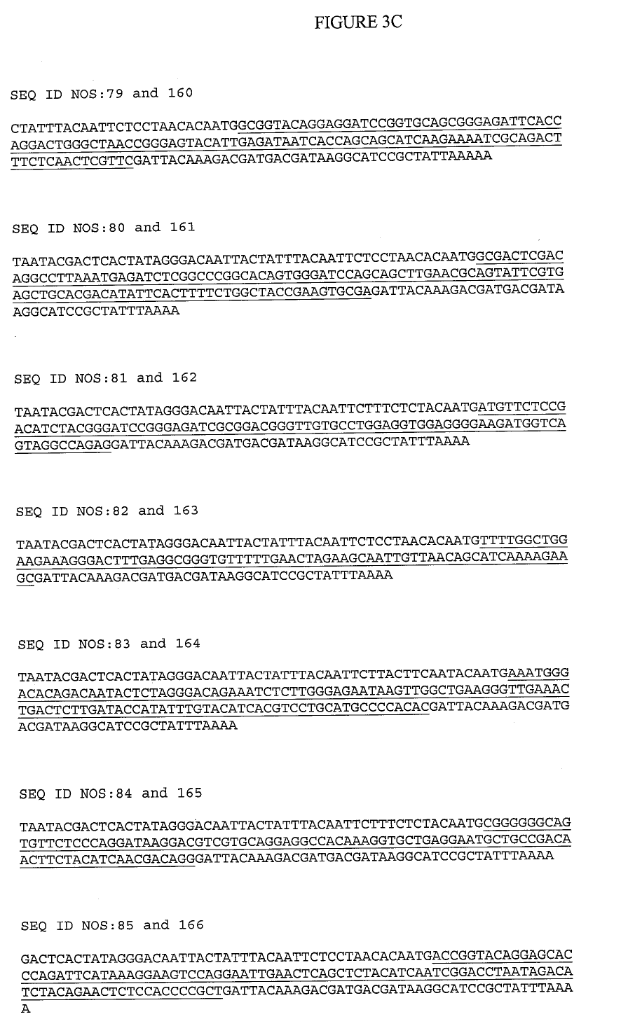

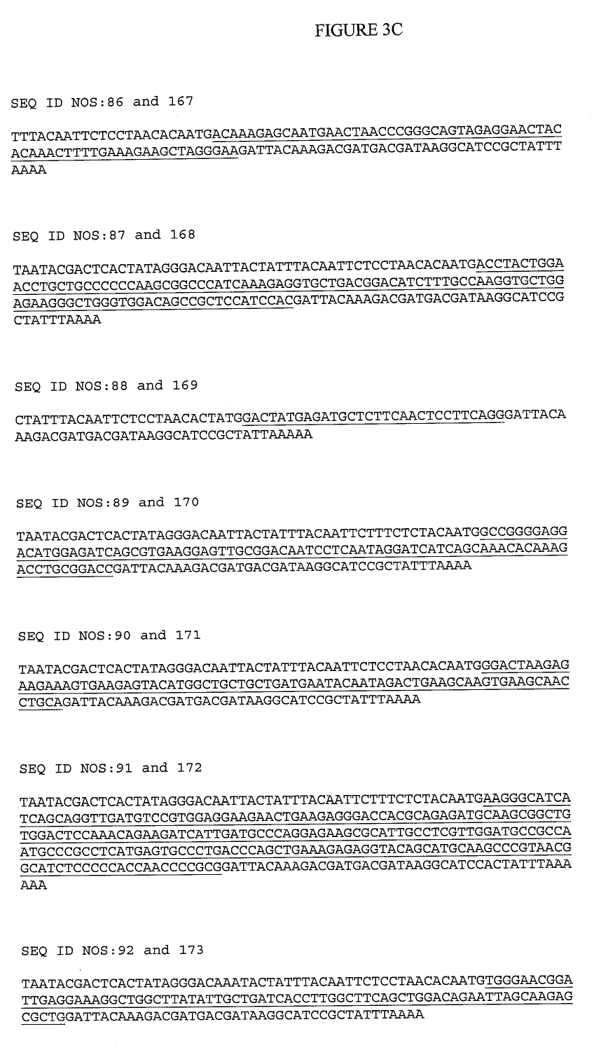

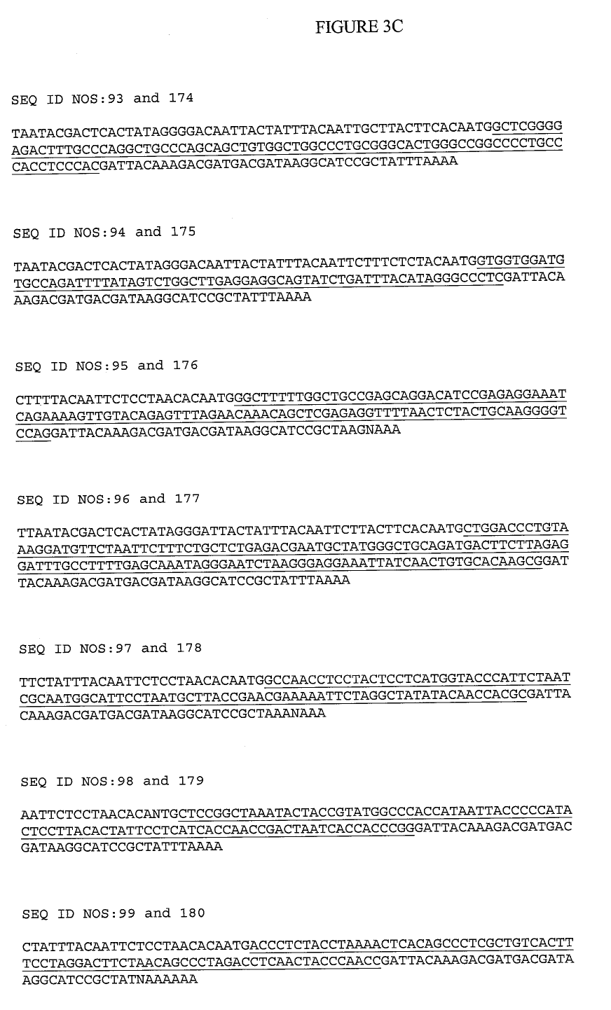

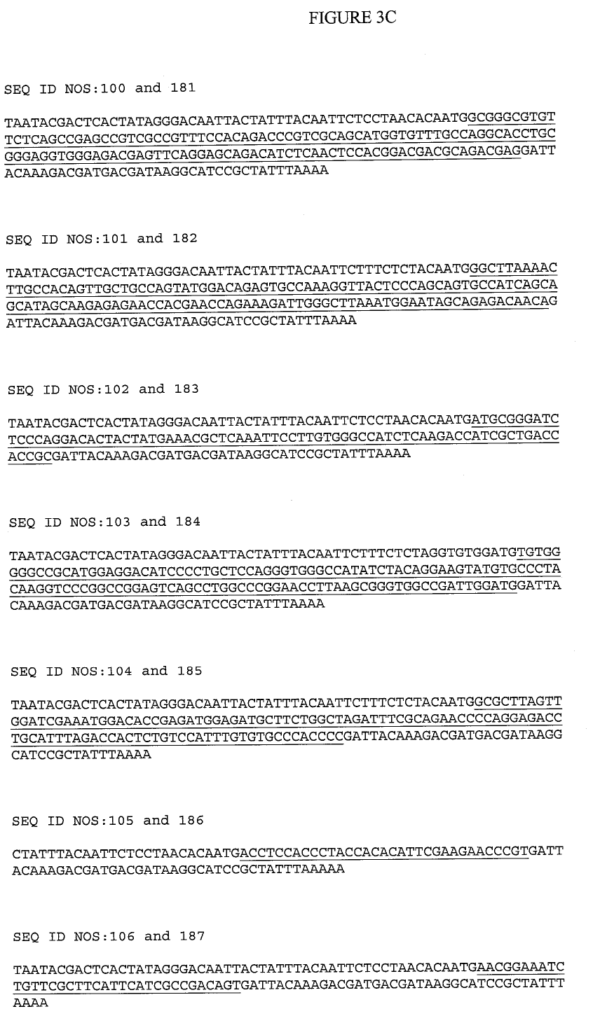

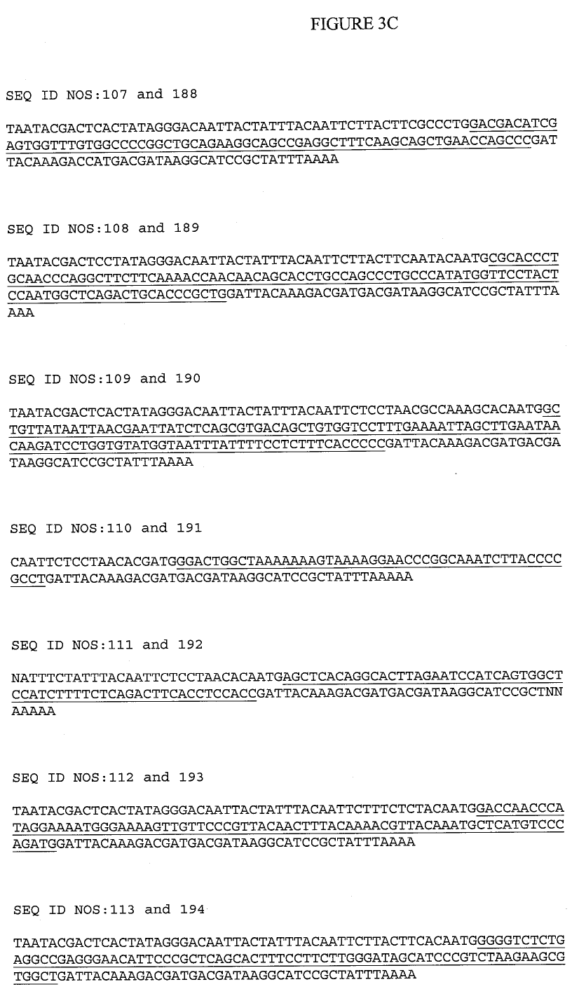

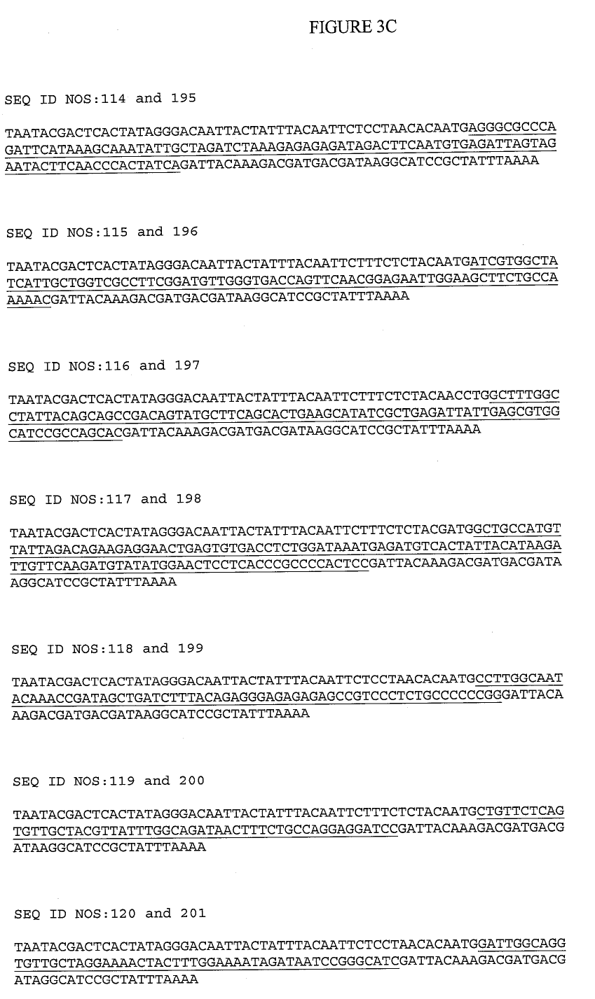

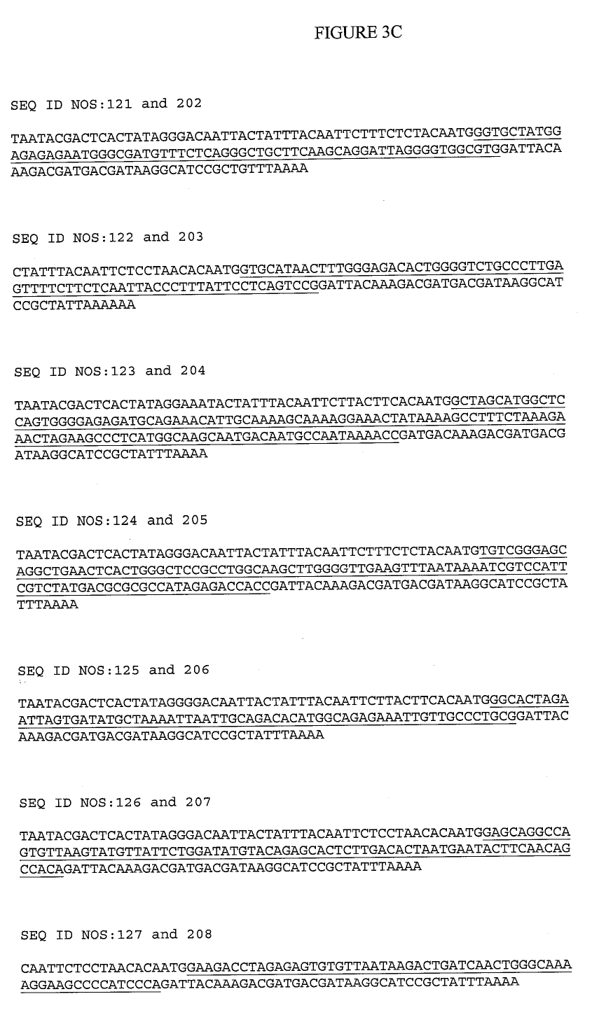

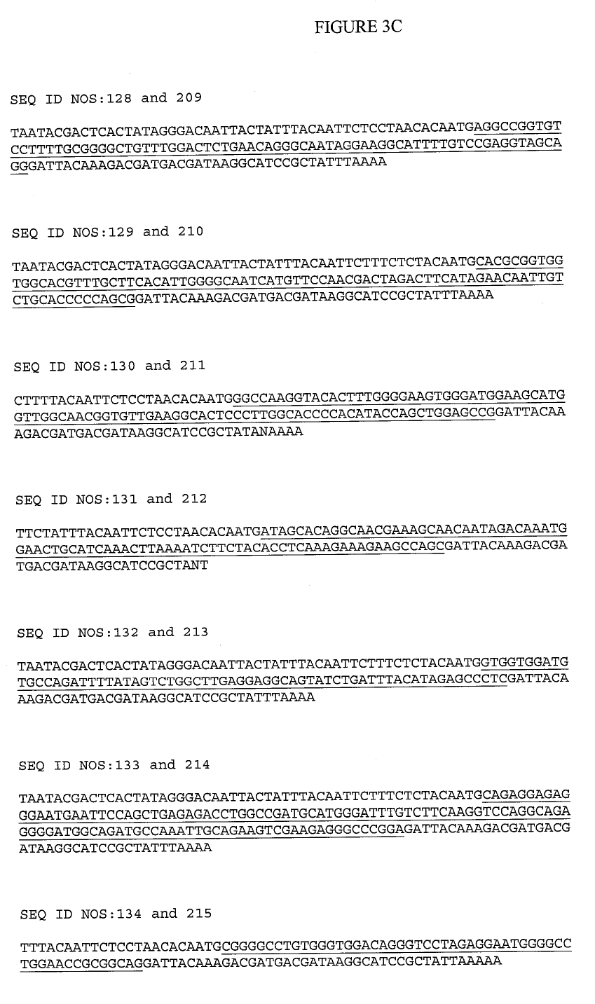

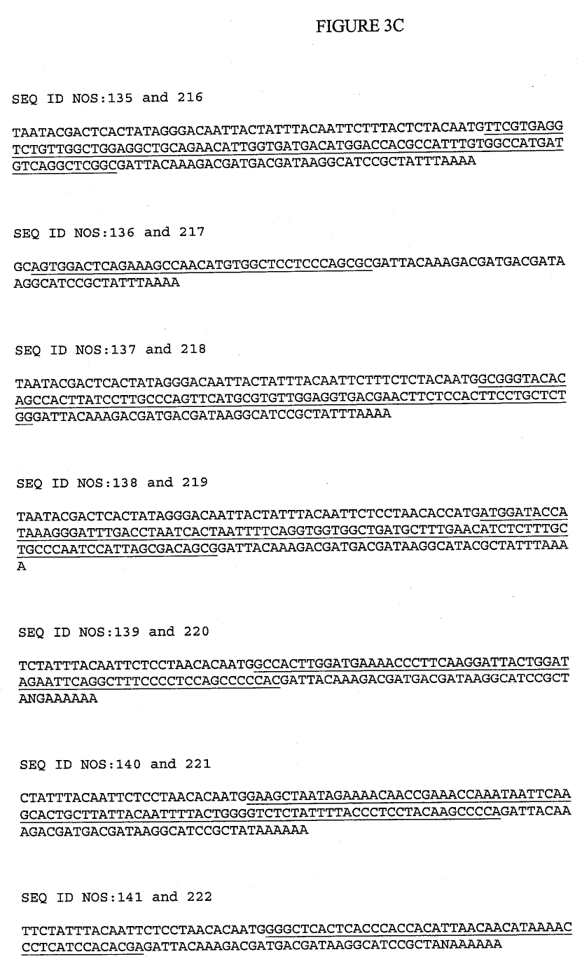

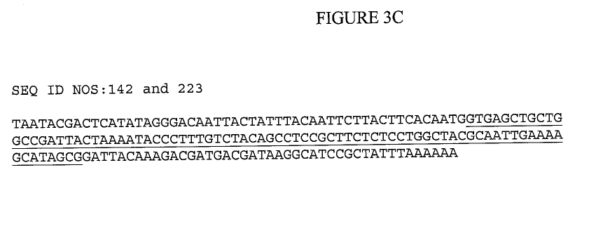

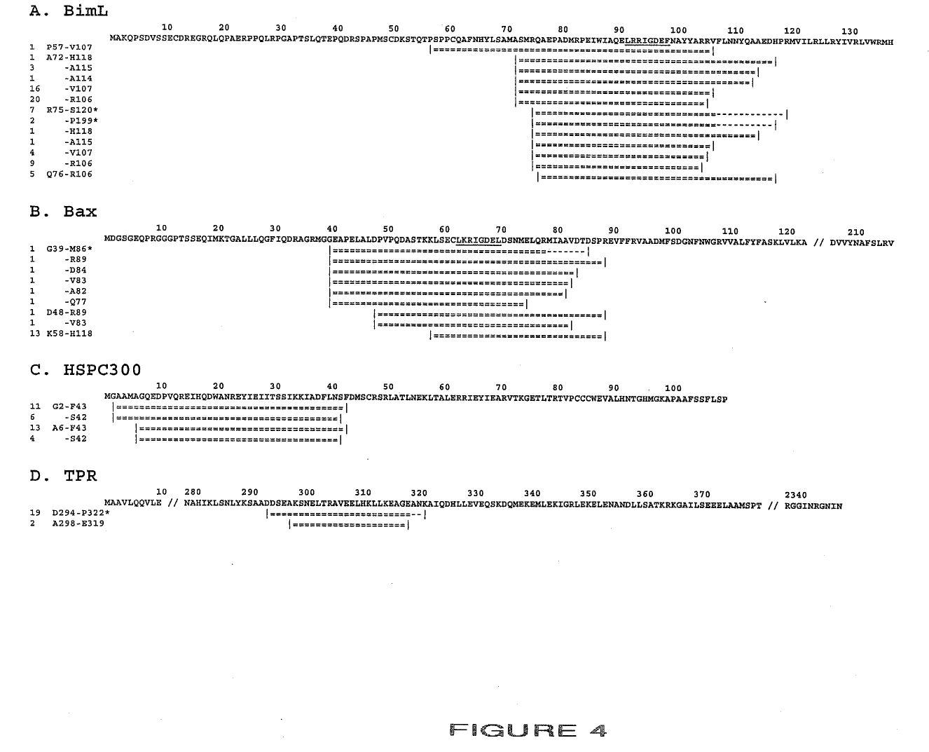

A total of 378 sequences were obtained from the above-described binding assay. Of the sequences, 181 were from the kidney library, 85 were from the liver library, and 112 were from the bone marrow library. Initial analysis of the sequences revealed a total of 71 distinct sequence clusters. Six of the clusters (8%) originated from all three libraries, 14 clusters (20%) originated from two of the three libraries, and the remaining 51 clusters (72%) originated from only one library. Many of the clusters contained a number of identical clones as well as a variety of clones with distinct 5′ or 3′ ends. This variety reflects the random priming used to prepare the library and allowed minimal functional regions of the Bcl-X_{L}-binding polypeptides to be delineated based on the overlapping regions of individual family members (FIG. 4). The sequences were then subjected to both nBLAST and pBLAST searches to identify the proteins represented by each cluster. Thirty-six of the clones were from known polypeptides (SEQ ID NOS: 1-28, 63-69, and 71), twenty-three of the clones were from hypothetical or unknown polypeptides whose nucleic acid sequences were found in the database (SEQ ID NOS: 29-50, and 70), and twelve clones were unique polypeptide sequences (SEQ ID NOS: 51-62). These Bcl-X_{L}-binding polypeptide sequences are shown in FIG. 3B, and their corresponding nucleic acid sequences are shown underlined in FIG. 3C.

Twenty of the most frequently found Bcl-X_{L}-binding polypeptides are provided in Table 1. The number of clones in each cluster was further broken down by the number containing the source label of each individual library (NF indicates none found among the clones sequenced). The identification number of the specific clone from each cluster chosen for further characterization is also indicated. The numbers present in Table 1 reflect the diversity of polypeptides that interact with other polypeptides attainable from large libraries generated by the in vitro methods of the invention.

TABLE 1
Frequently found BCl-X_{L} binding polypeptides

| | Kidney | Liver | Marrow | Total | Clone |
|---|---|---|---|---|---|
| Protein | (181) | (85) | (112) | (378) | ID |
| Bim | 43 | 11 | 36 | 90 | T44 |
| HSPC300 | 9 | 15 | 11 | 35 | C68 |
| TPR, nuclear pore | 23 | NF | NF | 23 | C55 |
| complex-associated protein | | | | | |
| Bax | 19 | NF | 3 | 22 | C49 |
| Novel Protein A | 1 | 11 | 6 | 18 | V18 |
| cDNA FLJ23277, Clone | 12 | 2 | 2 | 16 | X42 |
| HEP03322 | | | | | |
| Hypothetical protein | NF | 1 | 15 | 16 | V47 |
| DKFZp586HO623 | | | | | |
| Syntaxin 4A | 8 | 4 | NF | 12 | U58 |
| Tumor protein HDCMB21P | 1 | 5 | 5 | 11 | V50 |
| Proline/Glutamine rich splicing | 7 | 1 | 1 | 9 | — |
| factor | | | | | |
| Novel Protein B | 3 | 5 | NF | 8 | V68 |
| Talin | 4 | NF | 1 | 5 | X56 |
| Thyroid hormone | 5 | NF | NF | 5 | U25 |
| receptor-associated protein | | | | | |
| Sterol regulatory element | NF | NF | 5 | 5 | W17 |
| binding txn factor | | | | | |
| Bcl-2 related proline-rich | NF | 2 | 3 | 5 | Y75 |
| protein BPR | | | | | |
| cDNA FLJ22171, clone | NF | NF | 5 | 5 | T42 |
| HRC00654 | | | | | |
| Toll-like receptor 3 | 4 | NF | NF | 4 | U15 |
| Calpain | 1 | 3 | NF | 4 | V53 |
| Bak | 2 | 1 | NF | 3 | C32 |
| Novel protein D | NF | 1 | 2 | 3 | T25 |

The most abundant Bcl-X_{L}-binding polypeptide (˜25% of the total) was that of Bim, which was originally identified as a partner of Bcl-2 in a protein interaction screen and subsequently shown to bind to Bcl-X_{L} (O'Connor et al., EMBO J. 17:384-395, 1998). Two other proteins out of the top twenty, Bak and Bax, contain BH3 domains known to interact with Bcl-X_{L} (Diaz et al., supra). A fourth member of the Bcl-2 family, BPR, was also found in this screen. This newly reported member of the Bcl-2 family was not present in the database during the initial search. That a protein that was initially categorized as unknown is indeed a member of the Bcl-2 family reinforces the hypothesis that other novel polypeptides identified in the screen may also be members of the Bcl-2 family. While initial reports indicate that BPR contains a BH2 domain (Scorilas et al., unpublished, 2000), the present invention indicates that it also contains a BH3 domain.

Further analysis of the known Bcl-X_{L}-binding polypeptides was done to determine whether each selected Bcl-X_{L}-binding polypeptide sequence was from the coding region or UTR and if the reading frame matched that of the native protein. This analysis was used as a filter to eliminate false positives; polypeptides that failed at this step were not further characterized. Twenty-seven out of the thirty-six clusters from known polypeptides were in frame and within their native ORFs. Three out of thirty-six, proline/glutamine rich splicing factor (SEQ ID NO: 63), UDP glucoronosyl transferase 2B4 precursor (SEQ ID NO: 71), and cDNA FLJ20617 (SEQ ID NO: 70) were from the incorrect reading frame. Two clusters, transforming growth factor and arsenate resistance protein (SEQ ID NOS: 64 and 66, respectively), had inserts in the reversed orientation relative to the parent mRNA and probably arose due either to incomplete removal of the first strand primer after cDNA synthesis or re-priming on the cDNA strand after first strand synthesis. An additional four clusters were derived from reportedly noncoding regions of the parent mRNA, that is, the 3′ UTR (L-plastin, K-ras oncogene, lysosomal pepstatin insensitive protease, and MYBPC3; SEQ ID NOS: 65, 67, 68, and 69, respectively).

FIG. 4 shows an alignment of selected Bcl-X_{L}-binding polypeptides with their parental proteins, identified as described above. Each unique fragment was analyzed to determine the location of the amino and carboxyl termini within the parental protein sequence and these amino acids are indicated by residue and number. The number of isolated clones corresponding to each unique fragment was determined and is indicated next to the fragment ID. These fragments are mapped against the parental sequences of BimL, Bax, HSPC300, and TPR.

Affinity and Specificity of the Bcl-X_{L} Binding Polypeptides

The initial sequencing data showed the relative frequency of each clone in the selected pool. Additional ranking of individual clones may provide valuable insight into the biological relevance of each interaction. For example, a binding affinity consistent with the cellular concentrations of the interacting proteins has been proposed as a litmus test for biological significance (Mayer, Mol. Biotechnol. 13:201-213, 1999). The great flexibility and precise control over assay conditions, such as target concentration and the presence of additives, is one of the advantages of the in vitro selection methods of the present invention. By ranking the selected polypeptides based on readily assayable characteristics, it is possible to quickly identify a subset of polypeptides for assays that address the in vivo activity of the identified polypeptides.

To determine the affinity of the selected Bcl-X_{L}-binding polypeptides, each cluster of selected sequences was aligned and the shortest sequence was generally chosen as representing the minimal binding domain for that particular cluster. It should be noted that this shortest fragment may represent only a partial binding sequence and longer fragments may bind with higher affinity. The chosen clones were prepared as free peptides and used in the binding assay described below.

Purified radioactively labeled protein from the individual clones was incubated with immobilized Bcl-X_{L}-GST for one hour and, after washing, the amount bound was determined by scintillation counting. The binding at each concentration was normalized to that at the highest concentration and plotted versus concentration. FIG. 5 is a representational plot of the results of this binding assay. A selected Bak fragment (MGQVGRQLAIIGDDINRDYKDDDDKASA; SEQ ID NO: 152), containing a FLAG-M2 epitope, was synthetically produced as a free protein and used in a binding assay in which the concentration of immobilized Bcl-X_{L}-GST was varied from 11 nM to 28 μM. The amount of peptide bound to Bcl-X_{L} was determined by scintillation counting and normalized to that bound at the highest concentration. Normalized binding was then plotted versus Bcl-X_{L} concentration and fit to a binding curve using nonlinear regression. In this assay, all of the clones except one showed binding that was clearly dependent on target concentration. However, only binding curves that gave a high correlation coefficient ® value) were used to determine an affinity.

Binding affinities of the free Bcl-X_{L}-binding polypeptides (i.e., Bcl-X_{L}-binding polypeptides that are not part of fusions) ranged from approximately 2 nM to 10 μM, demonstrating the great range of affinities accessible by in vitro selection. The twenty clones with the highest affinity are presented in Table 2. The indicated clone from each sequence cluster was produced in vitro and the relative K_{d} was determined for binding to Bcl-X_{L}. The total number of clones in that sequence cluster is indicated for comparison of affinity to abundance.

TABLE 2
High Affinity Bcl-X_{L}-binding polypeptides

| clone | | Accession | K_{d} | |
|---|---|---|---|---|
| ID | Protein | Number | (μM) | Total clones |
| T44 | Bim | NP_006529 | 0.002 | 90 |
| T95 | Neutrophil cytosolic factor 2 | NP_000424 | 0.00416 | 2 |
| V47 | Hypothetical protein DKFZp586ho623 | NM_017540 | 0.0129 | 16 |
| C21 | Novel protein I | — | 0.07 | 3 |
| V18 | Novel protein A | — | 0.086 | 3 |
| X56 | Talin (splice variant) | NP_006280 | 0.093 | 6 |
| V72 | unknown protein from clone 425C14 on chrom. 6q22 | Z99129 | 0.28 | 1 |
| C32 | Bak | NP_001179 | 0.402 | 3 |
| Y37 | unknown protein from cDNA: FLJ21691, clone | AK025344 | 0.41 | 1 |
| | COL09555 | | | |
| Y75 | Bcl-2 related protein BPR | AF289220 | 0.42 | 5 |
| V06 | Golgi SNAP receptor complex member 1 | NP_004862 | 0.467 | 1 |
| C68 | HSCP300 | AF161418 | 0.58 | 35 |
| U58 | Syntaxin | NP_004595 | 0.64 | 12 |
| V50 | Tumor protein HDCMB21P | NP_003286 | 0.69 | 11 |
| C49 | Bax | NP_001179 | 0.76 | 22 |
| U15 | Toll-like receptor 3 | NP_003256 | 0.781 | 4 |
| Y01 | unknown protein from clone RP11-517O1 on chrom. X | AL355476 | 1.03 | 1 |
| W06 | Voltage dependent anion channel 3 | NP_005653 | 1.12 | 1 |
| V68 | Novel protein B | — | 1.16 | 8 |
| T25 | Novel protein D | — | 1.61 | 3 |

A comparison of K_{d} values of the Bcl-X_{L}-binding polypeptides (Table 2) to their frequency in the pool (Table 1) showed a 65% overlap; of the twenty lowest Bcl-X_{L}-binding polypeptide K_{d} values, thirteen were found within the top twenty most abundant Bcl-X_{L}-binding polypeptides, indicating a correlation between K_{d} and frequency. Five of the Bcl-X_{L}-binding polypeptides from the group with the twenty lowest K_{d} values, however, were observed only a single time, emphasizing the importance of post-selection characterization. Thus, the final representation of any given polypeptide within the selected pool may be determined by a number of factors: its abundance within the initial mRNA population used to prepare the library; the sum of efficiencies at each step in the mRNA display process (PCR, transcription, translation, fusion, etc.); and its affinity to the target.

As the target used in this selection was a GST fusion protein of Bcl-X_{L}, the specificity of each selected polypeptide was also tested by binding it to immobilized GST. The vast majority of Bcl-X_{L}-binding polypeptides exhibited background levels of binding (less than 2%) to GST. Of the eight proteins that bound more than 2% to GST, five bound eight to ten fold higher to the Bcl-X_{L}-GST fusion protein and so were deemed specific. The three remaining proteins bound poorly to the Bcl-X_{L}-GST fusion relative to GST alone and so were deemed non-specific.

Many Bcl-X_{L}-Binding Polypeptides Bind to the BH3 Domain of Bcl-X_{L}

As described above, the Bcl-2 family of proteins has been shown to form homo- and hetero-dimers through the binding of the BH3 domain of one protein in the corresponding binding pocket on its partner. Only three of the selected proteins (Bim, Bak, and Bax) were previously known to contain a BH3 domain. In order to determine if the other proteins bound to the BH3 domain binding site on Bcl-X_{L}, a competition assay was performed. The Bak BH3 domain peptide used as a positive control was prepared by chemical synthesis and used to compete with individual Bcl-X_{L}-binding polypeptides in a Bcl-X_{L} binding assay. The effectiveness of this competition was demonstrated in a titration of competitor concentration (FIG. 6). At a fixed concentration of immobilized Bcl-X_{L}, the Bak BH3 domain-containing peptide MGQVGRQLAIIGDDINRDYKDDDDKASA (SEQ ID NO: 152), also containing a FLAG-M2 epitope, was added at the indicated concentration along with a trace amount of a selected Talin fragment. After binding for 1 hour, the unbound material was removed and the bound protein was quantitated. The bound protein was assayed by scintillation counting, normalized to that bound in the absence of competitor, and plotted versus competitor concentration.

A competition assay was performed for each of the selected Bcl-X_{L}-binding polypeptides using 20 μM Bak BH3 competitor based on the titration shown in FIG. 6. Due to poor competition with the Bcl-X_{L}-binding polypeptides having the lowest K_{d} values (as determined above) a second competition was performed for some of these polypeptides using 100 μM competitor (FIG. 3A). Each Bcl-X_{L}-binding polypeptide was incubated with immobilized Bcl-X_{L} in the presence of competitor and the amount bound was normalized to a comparable reaction without competitor (FIG. 3A; see column labeled BakBH3 effect).

The Bcl-X_{L}-binding polypeptides were competed by the Bak BH3 domain, indicating that they probably bind at the same site on Bcl-X_{L}. The alternative explanation, a decrease in binding of the selected polypeptide at one site, due to a change in conformation of the target Bcl-X_{L} upon binding the competitor at a different site, was not tested in this assay. Only three of the selected proteins (clone x42, encoding SEQ ID NO: 35; clone t53, encoding SEQ ID NO: 25; and clone and w75, encoding SEQ ID NO: 37) were not competed at all by the BH3 domain, indicating that they may bind to a different site on Bcl-X_{L}.

Alignment of Selected Bcl-X_{L}-Binding Polypeptides

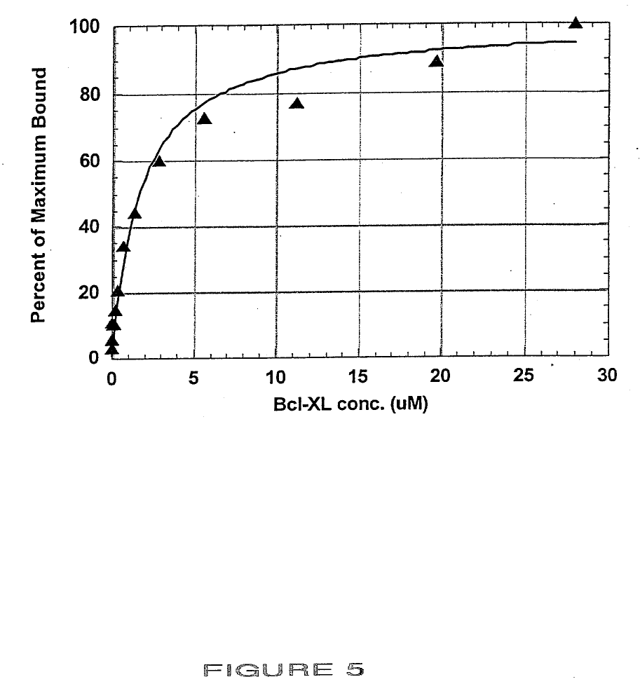

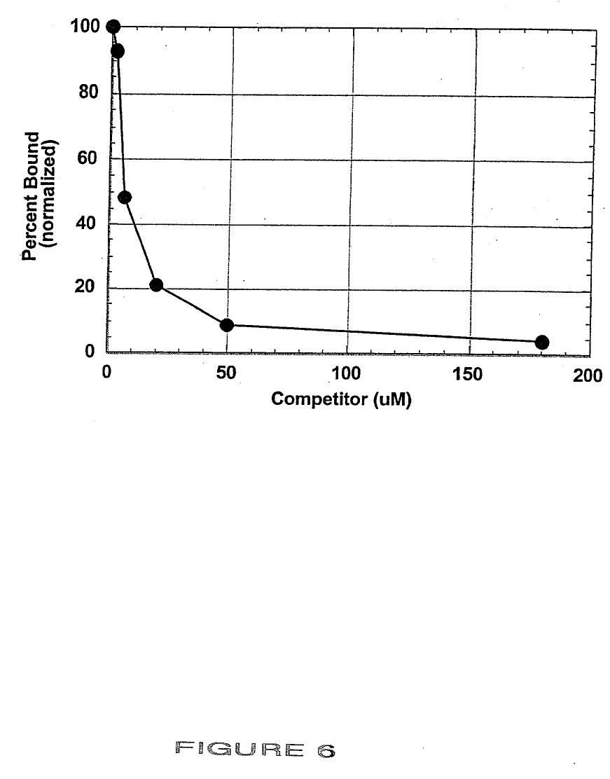

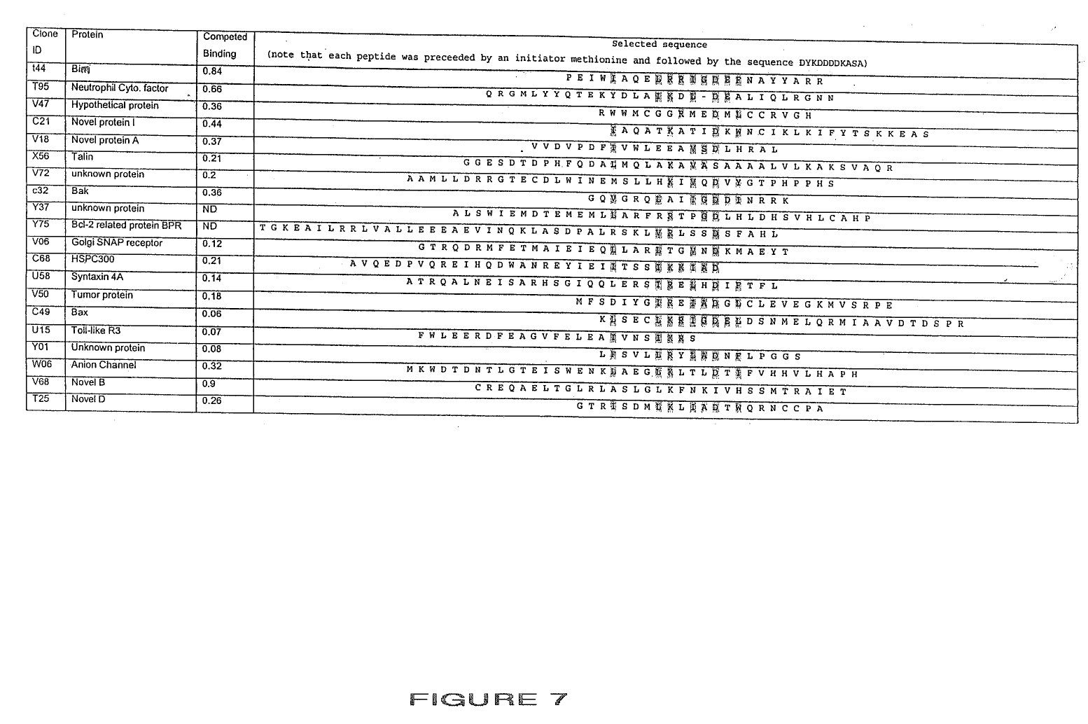

Competition for binding with the Bak BH3 domain indicated that most of the Bcl-X_{L}-binding polypeptides that were selected were binding at the same site. Therefore, each of the polypeptides was examined for the presence of a BH3 domain sequence. A tentative assignment could be made for most polypeptides. The Bcl-X_{L}-binding polypeptides with the highest affinity (Table 2) are shown in FIG. 7, aligned by sequence homology, where possible, to the known BH3 domains of Bim, Bak, and Bax. Most of the polypeptides have the hallmark periodicity of hydrophobic amino acids indicative of an amphipathic alpha helix. Additional homologies among the sequences are indicated by shading.

Additional Selection Experiments

Another selection to identify Bcl-X_{L}-GST fusion protein binders from mRNA display libraries prepared from tissue specific mRNAs of human bone marrow, brain, hippocampus, and thymus was initiated. Each library contained a unique 8 nucleotide source tag within the 5′ UTR to allow specific amplification of an individual library. The source tags AACTCCTC (SEQ ID NO: 246), AATCTACC (SEQ ID NO: 247), AACAACAC (SEQ ID NO: 248), and AATATTCC (SEQ ID NO: 249) were used for the libraries derived from mRNA from human bone marrow, brain hippocampus, and thymus, respectively. Prior to initiating the selection, the libraries were pooled.

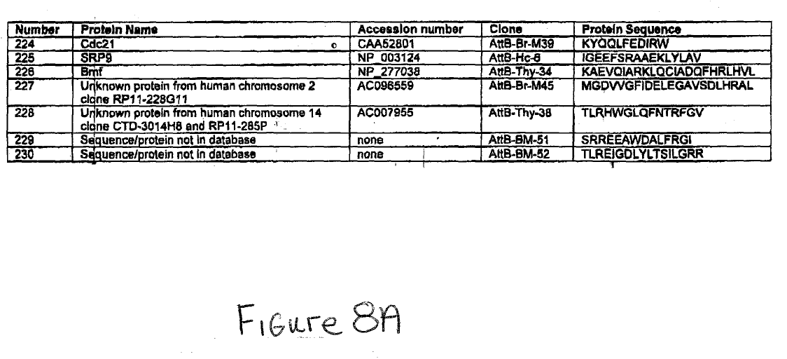

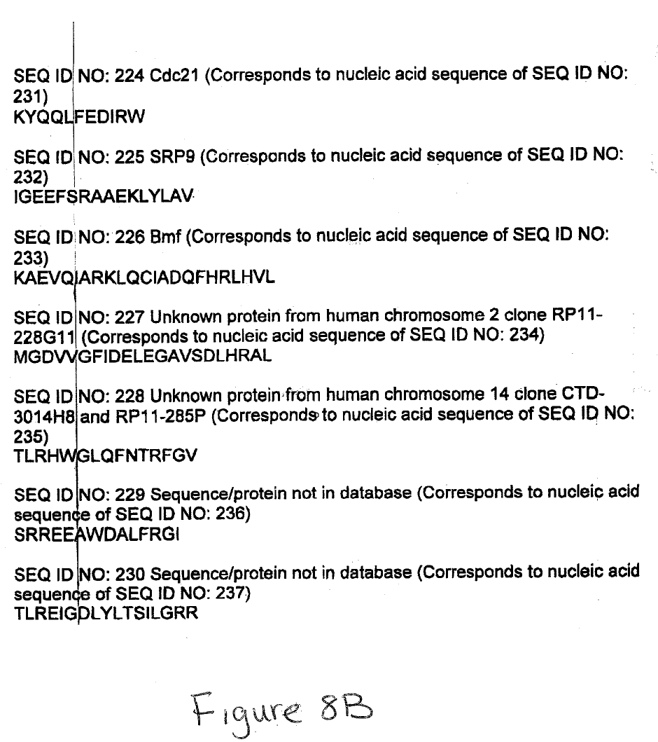

After five rounds of selection, each library was prepared individually after specific amplification using library specific primers and analyzed by cloning and sequencing. A total of 10 distinct sequence clusters were identified, of which 2 (Bim and Bax) were already identified in the previous selection. The unique sequences are shown in FIGS. 8A and 8B, and their corresponding nucleic acid sequences in FIG. 8C. Sequences of three of the clones were from known polypeptides (SEQ ID NOS: 224-226), sequences of two of the clones were from hypothetical or unknown polypeptides whose nucleic acid sequences were found in the database (SEQ ID: 227 and 228), and sequences of two of the clones were unique polypeptide sequences (SEQ ID: 229 and 230). All of the selected Bcl-X_{L}-binding polypeptide sequences were from the coding region of the native protein.

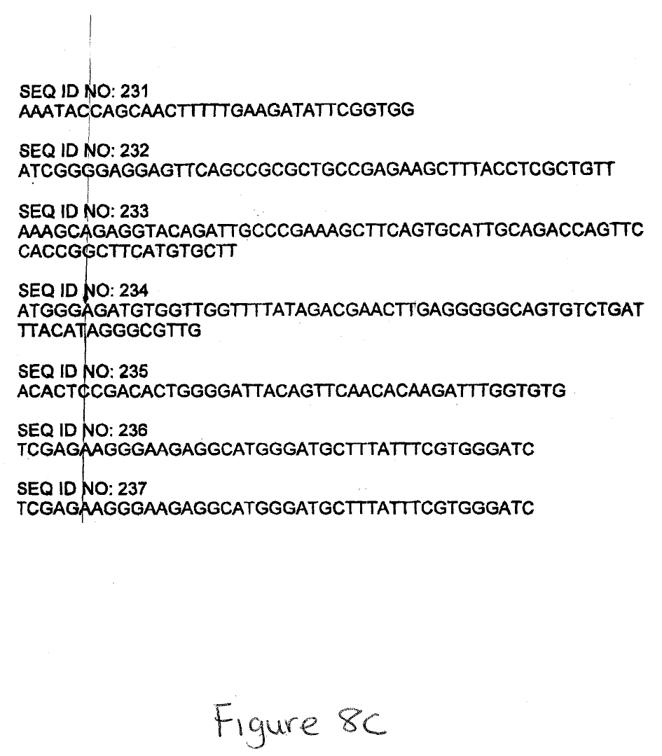

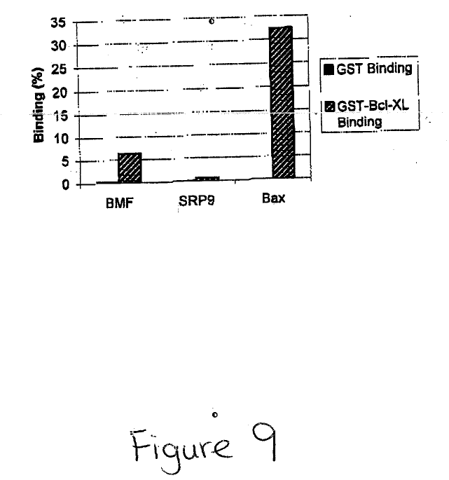

The following selected polypeptides that interacted with the Bcl-X_{L}-GST fusion protein were synthesized and purified as described: SRP9 (clone AttB-Hc-6) and Bmf (clone AttB-Thy-34), which were unique to this selection and Bax (clone AttB-Hc-7) as a positive control for binding to the Bcl-X_{L}-GST fusion protein. The purified polypeptides were assayed for binding to GST and to the Bcl-X_{L}-GST fusion protien (FIG. 9). Binding of Bax to the Bcl-X_{L}-GST fusion protein was the most efficient (32%), followed by Bmf (6%) and SRP9 (0.65%). Binding of all three purified polypeptides to GST were very low, with binding percentages not higher than 0.25%.

High-Throughput Identification of Protein-Protein Interactions

All of the procedures described above were essentially microcentrifuge tube based. Such systems are readily scalable through the use of microtiter techniques and are amenable to automation. In addition, the relatively laborious step of sequencing can be supplemented or replaced by array-based analysis of the pool, using, for example, Gene Discovery Arrays/Life Grids (Incyte Genomics, Palo Alto, Calif.) according to the manufacturer's instructions. These modifications to mRNA display technology enable its application to high-throughput, genome-wide identification of protein-protein interactions.

Cloning Full Length Nucleic Acid Molecules Encoding Bcl-X_{L}-Binding Polypeptides

Nucleic acid molecules encoding the full length polypeptide sequences of the identified Bcl-X_{L}-binding polypeptides can readily be cloned using standard hybridization or PCR cloning techniques and DNA from the source (as determined by the source label), for example, as described in Ausubel et al. (supra). An exemplary method for obtaining the full length polypeptide sequences employs, a standard nested PCR strategy that can be used with gene-specific (obtained from the nucleic acid sequence encoding the Bcl-X_{L}-binding polypeptide) and flanking adaptors from double stranded cDNA prepared from the source of the identified Bcl-X_{L}-binding polypeptide. In addition, 5′ flanking sequence can be obtained using 5′ RACE techniques known to those of skill in the art.

Synthesis of Bcl-X_{L}-Binding Polypeptides

Additional characteristics of the Bcl-X_{L}-binding polypeptides may be analyzed by synthesizing the polypeptides in various cell types or in vitro systems. The function of Bcl-X_{L}-binding polypeptides may then be examined under different physiological conditions. Alternatively, cell lines may be produced which over-express the nucleic acid encoding a Bcl-X_{L}-binding polypeptide, allowing purification of a Bcl-X_{L}-binding polypeptide for biochemical characterization, large-scale production, antibody production, or patient therapy.

For polypeptide expression, eukaryotic and prokaryotic expression systems may be generated in which nucleic acid sequences encoding Bcl-X_{L}-binding polypeptides are introduced into a plasmid or other vector, which is then used to transform living cells. Constructs in which the nucleic acid sequences are inserted in the correct orientation into an expression plasmid may be used for protein expression. Alternatively, portions of gene sequences encoding the Bcl-X_{L}-binding polypeptide, including wild-type or mutant Bcl-X_{L}-binding polypeptide sequences, may be inserted. Prokaryotic and eukaryotic expression systems allow various important functional domains of the Bcl-X_{L}-binding polypeptides to be recovered, if desired, as fusion proteins, and then used for binding, structural, and functional studies and also for the generation of appropriate antibodies. If Bcl-X_{L}-binding polypeptide expression induces terminal differentiation in some types of cells, it may be desirable to express the protein under the control of an inducible promoter in those cells.

Standard expression vectors contain promoters that direct the synthesis of large amounts of mRNA corresponding to the inserted nucleic acid encoding a Bcl-X_{L}-binding polypeptide in the plasmid-bearing cells. They may also include eukaryotic or prokaryotic origin of replication sequences allowing for their autonomous replication within the host organism, sequences that encode genetic traits that allow vector-containing cells to be selected in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria such as Escherichia coli requires the insertion of the nucleic acid sequence encoding a Bcl-X_{L}-binding polypeptide into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Once the appropriate expression vectors containing a nucleic acid sequence encoding a Bcl-X_{L}-binding polypeptide, or fragment, fusion, or mutant thereof, are constructed, they are introduced into an appropriate host cell by transformation techniques, including calcium phosphate transfection, DEAE-dextran transfection, electroporation, microinjection, protoplast fusion, and liposome-mediated transfection. The host cells that are transfected with the vectors of this invention may include (but are not limited to) E. coli or other bacteria, yeast, fungi, insect cells (using, for example, baculoviral vectors for expression), or cells derived from mice, humans, or other animals. Mammalian cells can also be used to express the Bcl-X_{L}-binding polypeptides using, for example, a vaccinia virus expression system described, for example, in Ausubel et al. (supra).

Expression of Bcl-X_{L}-binding polypeptides, fusions, polypeptide fragments, or mutants encoded by cloned DNA is also possible using, for example, the T7 late-promoter expression system. This system depends on the regulated expression of T7 RNA polymerase, an enzyme encoded in the DNA of bacteriophage T7. The T7 RNA polymerase initiates transcription at a specific 23-bp promoter sequence called the T7 late promoter. Copies of the T7 late promoter are located at several sites on the T7 genome, but none is present in E. coli chromosomal DNA. As a result, in T7-infected E. coli cells, T7 RNA polymerase catalyzes transcription of viral genes but not of E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system. The resulting protein can be radioactively labeled. Plasmid vectors containing late promoters and the corresponding RNA polymerases from related bacteriophages such as T3, T5, and SP6 may also be used for production of proteins from cloned DNA. E. coli can also be used for expression using an M13 phage such as mGPI-2. Furthermore, vectors that contain phage lambda regulatory sequences, or vectors that direct the expression of fusion proteins, for example, a maltose-binding protein fusion protein or a glutathione-S-transferase fusion protein, also may be used for expression in E. coli.

Eukaryotic expression systems are useful for obtaining appropriate post-translational modification of expressed polypeptides. Transient transfection of a eukaryotic expression plasmid allows the transient production of Bcl-X_{L}-binding polypeptides by a transfected host cell. Bcl-X_{L}-binding polypeptides may also be produced by a stably-transfected mammalian cell line. A number of vectors suitable for stable transfection of mammalian cells are available to the public (e.g., see Pouwels et al., Cloning Vectors: A Laboratory Manual, 1985, Supp. 1987), as are methods for constructing such cell lines (see e.g., Ausubel et al., supra). In one example, a nucleic acid molecule encoding a Bcl-X_{L}-binding polypeptide, fusion, mutant, or polypeptide fragment is cloned into an expression vector that includes the dihydrofolate reductase (DHFR) gene. Integration of the plasmid and, therefore, integration of the nucleic acid sequence encoding the Bcl-X_{L}-binding polypeptide into the host cell chromosome is selected for by inclusion of 0.01-300 μM methotrexate in the cell culture medium (as described, for example in Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al. (supra). These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR cells, ATCC Accession No. CRL 9096) are among those most preferred for DHFR selection of a stably-transfected cell line or DHFR-mediated gene amplification.

Eukaryotic cell expression of Bcl-X_{L}-binding polypeptides facilitates studies of the gene and gene products encoding Bcl-X_{L}-binding polypeptides, including determination of proper expression and post-translational modifications for biological activity, identifying regulatory elements located in the 5′, 3′, and intron regions of nucleic acid molecules encoding Bcl-X_{L}-binding polypeptides and their roles in tissue regulation of Bcl-X_{L}-binding polypeptide expression. It also permits the production of large amounts of normal and mutant proteins for isolation and purification, and the use of cells expressing Bcl-X_{L}-binding polypeptides as a functional assay system for antibodies generated against the protein. Eukaryotic cells expressing Bcl-X_{L}-binding polypeptides may also be used to test the effectiveness of pharmacological agents on apoptotic diseases or as means by which to study Bcl-X_{L}-binding polypeptides as components of a transcriptional activation system. Expression of Bcl-X_{L}-binding polypeptides, fusions, mutants, and polypeptide fragments in eukaryotic cells also enables the study of the function of the normal complete polypeptide, specific portions of the polypeptide, or of naturally occurring polymorphisms and artificially-produced mutated polypeptides. The DNA sequences encoding Bcl-X_{L}-binding polypeptides can be altered using procedures known in the art, such as restriction endonuclease digestion, DNA polymerase fill-in, exonuclease deletion, terminal deoxynucleotide transferase extension, ligation of synthetic or cloned DNA sequences, and site-directed sequence alteration using specific oligonucleotides together with PCR.

Another preferred eukaryotic expression system is the baculovirus system using, for example, the vector pBacPAK9, which is available from Clontech (Palo Alto, Calif.). If desired, this system may be used in conjunction with other protein expression techniques, for example, the myc tag approach described by Evan et al. (Mol. Cell Biol. 5:3610-3616, 1985).

Once the recombinant protein is expressed, it can be isolated from the expressing cells by cell lysis followed by protein purification techniques, such as affinity chromatography. In this example, an anti-Bcl-X_{L}-binding polypeptide antibody, which may be produced by the methods described herein, can be attached to a column and used to isolate the recombinant Bcl-X_{L}-binding polypeptides. Lysis and fractionation of Bcl-X_{L}-binding polypeptide-harboring cells prior to affinity chromatography may be performed by standard methods (see e.g., Ausubel et al. (supra). Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Polypeptides of the invention, particularly short Bcl-X_{L}-binding fragments, can also be produced by chemical synthesis (e.g., by the methods described in Solid Phase Peptide Synthesis, 2nd ed., 1984, The Pierce Chemical Co., Rockford, Ill.). These general techniques of polypeptide expression and purification can also be used to produce and isolate useful Bcl-X_{L}-binding polypeptide fragments or analogs, as described herein.

Those skilled in the art of molecular biology will understand that a wide variety of expression systems may be used to produce the recombinant Bcl-X_{L}-binding polypeptides. The precise host cell used is not critical to the invention. The Bcl-X_{L}-binding polypeptides may be produced in a prokaryotic host (e.g., E. coli) or in a eukaryotic host (e.g., S. cerevisiae, insect cells such as Sf9 cells, or mammalian cells such as COS-1, NIH 3T3, or HeLa cells). These cells are commercially available from, for example, the American Type Culture Collection, Rockville, Md. (see also Ausubel et al., supra). The method of transformation and the choice of expression vehicle (e.g., expression vector) will depend on the host system selected. Transformation and transfection methods are described, e.g., in Ausubel et al. (supra) and expression vehicles may be chosen from those provided, e.g., in Pouwels et al., Cloning Vectors: A Laboratory Manual, 1985, Supp. 1987.

In addition, prokaryotic and eukaryotic in vitro systems can be utilized for the generation of Bcl-X_{L}-binding polypeptides. Such methods are described, for example by Ausubel et al.(supra). Proteins can be synthesized using, for example, in vitro transcription and translation methods. Rabbit reticulocyte lysates, wheat germ lysates, or E. coli lysates can be used to translate exogenous mRNAs from a variety f eukaryotic and prokaryotic sources. Kits for the in vitro production of polypeptides are available, for example, from Ambion (Austin, Tex.).

Bcl-X_{L}-Binding Polypeptide Fragments

Polypeptide fragments that incorporate various portions of Bcl-X_{L}-binding polypeptides are useful in identifying the domains or amino acids important for the biological activities of Bcl-X_{L}-binding polypeptides, and the present invention helps to identify these critical domains (FIG. 4). Methods for generating such fragments are well known in the art (see, for example, Ausubel et al. (supra)) using the nucleotide sequences provided herein. For example, a Bcl-X_{L}-binding polypeptide fragment may be generated by PCR amplifying the desired fragment using oligonucleotide primers designed based upon the nucleic acid sequences encoding Bcl-X_{L}-binding polypeptides. Preferably, the oligonucleotide primers include unique restriction enzyme sites that facilitate insertion of the fragment into the cloning site of a mammalian expression vector. This vector may then be introduced into a mammalian cell by artifice by the various techniques known in the art and described herein, resulting in the production of a Bcl-X_{L}-binding polypeptide gene fragment.

Bcl-X_{L}-binding polypeptide fragments will be useful in evaluating the portions of the polypeptide involved in important biological activities, such as protein-protein interactions. These fragments may be used alone, or as chimeric fusion proteins. Bcl-X_{L}-binding polypeptide fragments may also be used to raise antibodies specific for various regions of Bcl-X_{L}-binding polypeptides. Any portion of the Bcl-X_{L}-binding polypeptide amino acid sequence may be used to generate antibodies.

Bcl-X_{L}-Binding Polypeptide Antibodies

In order to prepare polyclonal antibodies, Bcl-X_{L}-binding polypeptides, fragments of Bcl-X_{L}-binding polypeptides, or fusion polypeptides containing defined portions of Bcl-X_{L}-binding polypeptides may be synthesized in bacteria by expression of corresponding DNA sequences in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, and then coupled to a carrier protein and mixed with Freund's adjuvant (to enhance stimulation of the antigenic response in an innoculated animal) and injected into rabbits or other laboratory animals. Alternatively, protein can be isolated from Bcl-X_{L}-binding polypeptide-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from Bcl-X_{L}-binding polypeptide-expressing tissue electrophoretically fractionated on a polyacrylamide gel to identify Bcl-X_{L}-binding polypeptides. Alternatively, synthetic peptides can be made that correspond to the antigenic portions of the protein and used to innoculate the animals.

In order to generate a peptide for use in making, for example, Bcl-X_{L}-binding polypeptide-specific antibodies, a Bcl-X_{L}-binding polypeptide sequence may be expressed as a C-terminal fusion with glutathione S-transferase (GST; Smith et al., Gene 67:31-40, 1988). The fusion protein may be purified on glutathione-Sepharose beads, eluted with glutathione, cleaved with thrombin (at the engineered cleavage site), and purified to the degree required to successfully immunize rabbits. Primary immunizations may be carried out with Freund's complete adjuvant and subsequent immunizations performed with Freund's incomplete adjuvant. Antibody titers are monitored by Western blot and immunoprecipitation analyses using the thrombin-cleaved Bcl-X_{L}-binding polypeptide fragment of the Bcl-X_{L}-binding-GST fusion polypeptide. Immune sera are affinity purified using CNBr-Sepharose-coupled Bcl-X_{L}-binding polypeptide. Antiserum specificity may be determined using a panel of unrelated GST fusion proteins.

Alternatively, monoclonal Bcl-X_{L}-binding polypeptide antibodies may also be produced by using, as an antigen, a Bcl-X_{L}-binding polypeptide isolated from Bcl-X_{L}-binding polypeptide-expressing cultured cells or Bcl-X_{L}-binding polypeptide isolated from tissues. The cell extracts, or recombinant protein extracts containing Bcl-X_{L}-binding polypeptide, may, for example, be injected with Freund's adjuvant into mice. Several days after being injected, the mouse spleens are removed, the tissues are disaggregated, and the spleen cells are suspended in phosphate buffered saline (PBS). The spleen cells serve as a source of lymphocytes, some of which are producing antibody of the appropriate specificity. These are then fused with permanently growing myeloma partner cells, and the products of the fusion are plated into a number of tissue culture wells in the presence of a selective agent such as hypoxanthine, aminopterine, and thymidine (HAT). The wells are then screened by ELISA to identify those containing cells making antibody capable of binding a Bcl-X_{L}-binding polypeptide or polypeptide fragment or mutant thereof. These are then re-plated and after a period of growth, these wells are again screened to identify antibody-producing cells. Several cloning procedures are carried out until over 90% of the wells contain single clones that are positive for antibody production. From this procedure a stable line of clones that produce the antibody is established. The monoclonal antibody can then be purified by affinity chromatography using Protein A Sepharose, ion-exchange chromatography, as well as variations and combinations of these techniques. Truncated versions of monoclonal antibodies may also be produced by recombinant methods in which plasmids are generated that express the desired monoclonal antibody fragment(s) in a suitable host.

As an alternate or adjunct immunogen to GST fusion proteins, peptides corresponding to relatively unique hydrophilic regions of Bcl-X_{L}-binding polypeptide may be generated and coupled to keyhole limpet hemocyanin (KLH) through an introduced C-terminal lysine. Antiserum to each of these peptides is similarly affinity-purified on peptides conjugated to BSA, and specificity is tested by ELISA and Western blotting using peptide conjugates, and by Western blotting and immunoprecipitation using Bcl-X_{L}-binding polypeptide, for example, expressed as a GST fusion protein.

Alternatively, monoclonal antibodies may be prepared using the Bcl-X_{L}-binding polypeptides described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, New York, N.Y., 1981; and Ausubel et al. (supra)). Once produced, monoclonal antibodies are also tested for specific Bcl-X_{L}-binding polypeptide recognition by Western blot or immunoprecipitation analysis (by the methods described in Ausubel et al., supra).

Monoclonal and polyclonal antibodies that specifically recognize a Bcl-X_{L}-binding polypeptide (or fragments thereof), such as those described herein, are considered useful in the invention. Antibodies that inhibit the activity of a Bcl-X_{L}-binding polypeptide described herein may be especially useful in preventing or slowing the development of a disease caused by inappropriate expression of a wild type or mutant Bcl-X_{L}-binding polypeptide.

Antibodies of the invention may be produced using Bcl-X_{L}-binding amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson and Wolf(CABIOS 4:181, 1988). These fragments can be generated by standard techniques, e.g., by PCR, and cloned into the pGEX expression vector (Ausubel et al., supra). GST fusion proteins are expressed in E. coli and purified using a glutathione-agarose affinity matrix as described in Ausubel et al., supra). To generate rabbit polyclonal antibodies, and to minimize the potential for obtaining antisera that is non-specific, or exhibits low-affinity binding to a Bcl-X_{L}-binding polypeptide, two or three fusions are generated for each protein, and each fusion is injected into at least two rabbits. Antisera are raised by injections in series, preferably including at least three booster injections.

In addition to intact monoclonal and polyclonal anti-Bcl-X_{L}-binding polypeptide antibodies, the invention features various genetically engineered antibodies, humanized antibodies, and antibody fragments, including F(ab′)2, Fab′, Fab, Fv, and sFv fragments. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also features of the invention (Green et al., Nature Genetics 7:13-21, 1994).

Ladner (U.S. Pat. Nos. 4,946,778 and 4,704,692) describes methods for preparing single polypeptide chain antibodies. Ward et al. (Nature 341:544-546, 1989) describe the preparation of heavy chain variable domains, which they term “single domain antibodies,” that have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describe various methods for producing immunoglobulins, and immunologically functional fragments thereof, which include at least the variable domains of the heavy and light chain in a single host cell. Cabilly et al. (U.S. Pat. No. 4,816,567) describe methods for preparing chimeric antibodies.

Affinity reagents or polypeptides from randomized polypeptide libraries that bind tightly to a desired polypeptides, for example, Bcl-X_{L}-binding polypeptides, fragments of Bcl-X_{L}-binding polypeptides, or fusion polypeptides containing defined portions of Bcl-X_{L}-binding polypeptides can also be obtained, using methods known to one skilled in the art. In addition, polypeptide affinity scaffolds may be used to bind a polypeptide of interest or to identify or optimize a polypeptide that binds to a polypeptide of interest, for example, Bcl-X_{L}-binding polypeptides, fragments of Bcl-X_{L}-binding polypeptides, or fusion polypeptides containing defined portions of Bcl-X_{L}-binding polypeptides. Such methods are described for example by Lipovsek et al. (WO 00/34784), hereby incorporated by reference.

Identification of Additional Bcl-X_{L}-Binding Polypeptide Genes

Standard techniques, such as the polymerase chain reaction (PCR) and DNA hybridization, may be used to clone Bcl-X_{L}-binding polypeptide homologues in other species and Bcl-X_{L}-binding polypeptide-related genes in humans. Bcl-X_{L}-binding-polypeptide-related genes and homologues may be readily identified using low-stringency DNA hybridization or low-stringency PCR with human Bcl-X_{L}-binding polypeptide probes or primers. Degenerate primers encoding human Bcl-X_{L}-binding polypeptides or human Bcl-X_{L}-binding polypeptide-related amino acid sequences may be used to clone additional Bcl-X_{L}-binding polypeptide-related genes and homologues by RT-PCR.

Alternatively, additional Bcl-X_{L}-binding polypeptides can be identified by utilizing consensus sequence information for Bcl-X_{L}-binding polypeptides to search for similar polypeptides. For example, polypeptide databases can be searched for proteins with the amphipathic alpha helix motif described above in Example 7. Candidate polypeptides containing such a motif can then be tested for their Bcl-X_{L}-binding properties, using methods described herein.

Assays for Compounds that Modulate Bcl-X_{L}-Binding Polypeptide Biological Activity

Bcl-X_{L}-binding polypeptide biological activity may be modulated in a number of different ways. For example, cellular concentrations of Bcl-X_{L}-binding polypeptides of can be altered, which would, in turn, affect overall Bcl-X_{L}-binding polypeptide biological activity. This is achieved, for example, by administering to a cell a compound that alters the concentration and/or activity of a Bcl-X_{L}-binding polypeptide.

We have shown herein that a number of polypeptides bind a Bcl-X_{L} polypeptide. Accordingly, compounds that modulate Bcl-X_{L}-binding polypeptide biological activity may be identified using any of the methods, described herein (or any analogous method known in the art), for measuring protein-protein interactions involving a Bcl-X_{L}-binding polypeptide. For example, the Bcl-X_{L}/Bcl-X_{L}-binding polypeptide assays described above may be used to determine whether the addition of a test compound increases or decreases binding activity of any (wild-type or mutant) Bcl-X_{L}-binding polypeptide to Bcl-X_{L}. A compound that increases or decreases the binding activity of a mutant Bcl-X_{L}-binding polypeptide may be useful for treating a Bcl-X_{L}-binding polypeptide-related disease, such as an apoptotic or proliferative disease. A compound that modulates Bcl-X_{L}-binding polypeptide biological activity may act by binding to either a Bcl-X_{L}-binding polypeptide or to Bcl-X_{L} itself, thereby reducing or preventing the biological activity of the Bcl-X_{L}-binding polypeptide.

Levels of Bcl-X_{L}-binding polypeptide may be modulated by modulating transcription, translation, or mRNA or protein turnover; such modulation may be detected using known methods for measuring mRNA and protein levels, e.g., RT-PCR and ELISA.

Test Compounds

In general, drugs for modulation of Bcl-X_{L}-binding polypeptide biological activity may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their Bcl-X_{L}-binding polypeptide-modulatory activities should be employed whenever possible.

When a crude extract is found to modulate (i.e., stimulate or inhibit) Bcl-X_{L}-binding polypeptide biological activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits Bcl-X_{L}-binding polypeptide biological activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases in which it is desirable to increase or decrease Bcl-X_{L}-binding polypeptide biological activity.

Construction of Transgenic Animals and Knockout Animals

Characterization of Bcl-X_{L}-binding polypeptide genes provides information that allows Bcl-X_{L}-binding polypeptide knockout animal models to be developed by homologous recombination. Similarly, animal models of Bcl-X_{L}-binding polypeptide overproduction may be generated by integrating one or more Bcl-X_{L}-binding polypeptide sequences into the genome, according to standard transgenic techniques. Moreover, the effect of Bcl-X_{L}-binding polypeptide gene mutations (e.g., dominant gene mutations) may be studied using transgenic mice carrying mutated Bcl-X_{L}-binding polypeptide transgenes or by introducing such mutations into the endogenous Bcl-X_{L}-binding polypeptide gene, using standard homologous recombination techniques.

Bcl-X_{L}-binding polypeptide knockout mice provide a tool for studying the role of Bcl-X_{L}-binding polypeptide in embryonic development and in disease. Moreover, such mice provide the means, in vivo, for testing therapeutic compounds for amelioration of diseases or conditions involving a Bcl-X_{L}-binding polypeptide-dependent or Bcl-X_{L}-binding polypeptide-affected pathway.

Construction of Polypeptide Knockout or Overexpressing Cell Lines

Characterization of Bcl-X_{L}-binding polypeptide genes also allows Bcl-X_{L}-binding polypeptide cell culture models to be developed, in which the Bcl-X_{L}-binding polypeptide is expressed or functions at a lower level than its wild-type counterpart cell. Such cell lines can be developed using standard antisense technologies. Similarly, cell culture models of Bcl-X_{L}-binding polypeptide overproduction or overactivation may be generated by integrating one or more Bcl-X_{L}-binding polypeptide sequences into the genome, according to standard molecular biology techniques. Moreover, the effect of Bcl-X_{L}-binding polypeptide gene mutations (e.g., dominant gene mutations) may be studied using cell cultures model in which the cells contain and overexpress a mutated Bcl-X_{L}-binding polypeptide.

Bcl-X_{L}-binding polypeptide knockout cells provide a tool for studying the role of Bcl-X_{L}-binding polypeptide in cellular events, including apoptosis. Moreover, such cell lines provide the cell culture means, for testing therapeutic compounds for modulation of the apoptototic pathway. Compounds that modulate apoptosis in these cell models can then be tested in animal models of diseases or conditions involving the apoptotic pathway.

### Other Embodiments

In other embodiments, the invention includes any polypeptide that is substantially identical to a Bcl-X_{L}-binding polypeptide; such homologues include other substantially pure naturally-occurring Bcl-X_{L}-binding polypeptides as well as natural mutants; induced mutants; DNA sequences that encode polypeptides and also hybridize to the nucleic acid sequence encoding a Bcl-X_{L}-binding polypeptide described herein under high stringency conditions or, less preferably under low stringency conditions (e.g., washing at 2× SSC at 40° C. with a probe length of at least 40 nucleotides); and proteins specifically bound by antisera directed to a Bcl-X_{L}-binding polypeptide. The invention also includes chimeric polypeptides that include a Bcl-X_{L}-binding polypeptide portion.

The invention further includes analogs of any naturally-occurring Bcl-X_{L}-binding polypeptide. Analogs can differ from the naturally-occurring Bcl-X_{L}-binding polypeptide by amino acid sequence differences, by post-translational modifications, or by both. Analogs of the invention will generally exhibit at least 85%, more preferably, 90%, and most preferably, 95% or even 99% identity with all or part of a naturally-occurring Bcl-X_{L}-binding polypeptide sequence. The length of sequence comparison is at least 5 amino acid residues, preferably, at least 10 amino acid residues, and more preferably, the full length of the polypeptide sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring Bcl-X_{L}-binding polypeptide by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs that contain residues other than L-amino acids, e.g., D-amino acids or non-naturally occurring or synthetic amino acids, e.g., β or γ amino acids.

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

What is claimed is:

1. A substantially pure human Bcl-X_{L}-binding polypeptide, said polypeptide consisting of the sequence of any of SEQ ID NOS: 4-50, 63-71, and 224-228.

2. A substantially pure human Bcl-X_{L}-binding polypeptide, said polypeptide comprising the sequence of any of SEQ ID NOS: 51-62, 229, and 230.

3. An isolated nucleic acid molecule encoding a polypeptide of claim 1 or 2.

4. The isolated nucleic acid of claim 3, wherein said nucleic acid molecule consists of the sequence of any of SEQ ID NOS: 156-202, 215-223, and 231-235.

5. The isolated nucleic acid of claim 3, wherein said nucleic acid molecule comprises the sequence of any of SEQ ID NOS: 203-214, 236, and 237.

6. A vector comprising the isolated nucleic acid molecule of claim 3.

7. A cell comprising the isolated nucleic acid molecule of claim 3.

8. A cell comprising the vector of claim 6.

9. A method of identifying a Bcl-X_{L}-binding polypeptide, said method comprising the steps of:
(a) providing a population of source labeled nucleic acid-protein fusion molecules;
(b) contacting said population of nucleic acid-protein fusion molecules with a Bcl-X_{L} polypeptide under conditions that allow interaction between the protein portion of a nucleic acid-protein fusion molecule of said population and said Bcl-X_{L} polypeptide;
(c) detecting an interaction between said protein portion and said Bcl-X_{L} polypeptide, thereby identifying a Bcl-X_{L}-binding polypeptide,

10. The method of claim 9, wherein said population of source labeled nucleic acid-protein fusion molecules is derived from more than one source.

11. The method of claim 9, wherein, in step (a), said nucleic acid-protein fusion molecules are detectably-labeled.

12. The method of claim 11, wherein, in step (b), said Bcl-X_{L} polypeptide is immobilized on a solid support; and wherein, in step (c), the detection of an interaction between said protein portion of a nucleic acid-protein fusion molecule and said Bcl-X_{L} polypeptide is carried out by detecting the labeled nucleic acid-protein fusion molecule bound to said solid support.

13. The method of claim 12, wherein said solid support is a chip or a bead.

14. A method of identifying a compound that modulates binding between a Bcl-X_{L} polypeptide and a Bcl-X_{L}-binding polypeptide, said method comprising the steps of:
(a) contacting a Bcl-X_{L} polypeptide with (i) a Bcl-X_{L}-binding polypeptide, said Bcl-X_{L}-binding polypeptide consisting of the sequence of any of SEQ ID NOS: 4-50, 63-71, and 224-228, and (ii) a candidate compound, under conditions that allow binding between said Bcl-X_{L} polypeptide and said Bcl-X_{L}-binding polypeptide;
(b) determining the level of binding between said Bcl-X_{L} polypeptide and said Bcl-X_{L}-binding polypeptide, wherein an increase or decrease in the level of binding between said Bcl-X_{L} polypeptide and said Bcl-X_{L}-binding polypeptide, relative to the level of binding between said Bcl-X_{L} polypeptide and said Bcl-X_{L}-binding polypeptide in the absence of said candidate compound, indicates a compound that modulates the binding between a Bcl-X_{L} polypeptide and a Bcl-X_{L}-binding polypeptide.

15. A method of identifying a compound that modulates binding between a Bcl-X_{L} polypeptide and a Bcl-X_{L}-binding polypeptide, said method comprising the steps of:
(a) contacting a Bcl-X_{L} polypeptide with (i) a Bcl-X_{L}-binding polypeptide, said Bcl-X_{L}-binding polypeptide comprising the sequence of any of SEQ ID NOS: 51-62, 229, and 230, and (ii) a candidate compound, under conditions that allow binding between said Bcl-X_{L} polypeptide and said Bcl-X_{L}-binding polypeptide;
(b) determining the level of binding between said Bcl-X_{L} polypeptide and said Bcl-X_{L}-binding polypeptide, wherein an increase or decrease in the level of binding between said Bcl-X_{L} polypeptide and said Bcl-X_{L}-binding polypeptide, relative to the level of binding between said Bcl-X_{L} polypeptide and said Bcl-X_{L}-binding polypeptide in the absence of said candidate compound, indicates a compound that modulates the binding between a Bcl-X_{L} polypeptide and a Bcl-X_{L}-binding polypeptide.

16. The method of claim 14 or 15, wherein said Bcl-X_{L}-binding polypeptide is part of a nucleic acid-protein fusion molecule.

17. The method of claim 14 or 15, wherein, in step (a), said Bcl-X_{L} polypeptide is attached to a solid support.

18. The method of claim 17, wherein said Bcl-X_{L}-binding polypeptide is detectably-labeled; and, in step (b), said level of binding between said Bcl-X_{L} polypeptide and said Bcl-X_{L}-binding polypeptide is determined by measuring the amount of Bcl-X_{L}-binding protein that binds to said solid support.

19. The method of claim 17, wherein said solid support is a chip or a bead.

20. A method of source-labeling a nucleic acid-protein fusion molecule, said method comprising the steps of:
(a) providing an RNA molecule;
(b) generating a first cDNA strand from said RNA molecule;
(c) generating a second cDNA strand complementary to said first cDNA strand, wherein said second cDNA strand comprises a nucleic acid sequence that identifies the source of said RNA molecule;
(d) generating an RNA molecule from the double stranded cDNA molecule of step (c)
(e) attaching a peptide acceptor to said RNA molecule of step (d);
(f) in vitro translating said RNA to generate a source labeled nucleic acid-protein fusion molecule.

21. A source-labeled nucleic acid-protein fusion molecule, said nucleic acid portion of said fusion molecule comprising a coding sequence for said protein and a label that identifies the source of said nucleic acid portion.

22. A method of identifying the source of the nucleic acid portion of a nucleic acid-protein fusion molecule, said method comprising the steps of:
(a) providing a population of nucleic acid-protein fusion molecules, said molecules comprising a source label that identifies the source of the nucleic acid portion of said nucleic acid-protein fusion molecules; and
(b) determining the identity of said source label, thereby identifying the source of the nucleic acid portion of a nucleic acid protein fusion molecule.

23. The method of claim 22, wherein said source label is cell type-specific.

24. The method of claim 22, wherein said source label is tissue-specific.

25. The method of claim 22, wherein said source label is species-specific.

26. The method of claim 22, wherein said population of nucleic acid-protein fusion molecules contains subpopulations of nucleic acid-protein fusion molecules from a plurality of sources.


SEQUENCE LISTING (253 sequences)

SEQ ID NO: 1 - protein, 35 aa, Homo sapiens
ASMRQAEPADMRPEIWIAQELRRIGDEFNAYYARE

SEQ ID NO: 2 - protein, 18 aa, Homo sapiens
GQVGRQLAIIGDDINRRK

SEQ ID NO: 3 - protein, 32 aa, Homo sapiens
KLSECLKRIGDELDSNMELQRMIAAVDTDSPR

SEQ ID NO: 4 - protein, 46 aa, Homo sapiens
TGKEAILRRLVALLEEEAEVINQKLASDPALRSKLVRLSSDSFAHL

SEQ ID NO: 5 - protein, 30 aa, Homo sapiens
QRGMLYYQTEKYDLAIKDLKEALIQLRGNN

SEQ ID NO: 6 - protein, 38 aa, Homo sapiens
GGESDTDPHFQDALMQLAKAVASAAAALVLKAKSVAQR

SEQ ID NO: 7 - protein, 35 aa, Homo sapiens
GTRQDRMFETMAIEIEQLLARLTGVNDKMAEYTNA

SEQ ID NO: 8 - protein, 33 aa, Homo sapiens
AVQEDPVQREIHQDWANREYIEIITSSIKKIAD

SEQ ID NO: 9 - protein, 33 aa, Homo sapiens
ATRQALNEISARHSGIQQLERSIRELHDIFTFL

SEQ ID NO: 10 - protein, 28 aa, Homo sapiens
MFSDIYGIREIADGLCLEVEGKMVSRPE

SEQ ID NO: 11 - protein, 25 aa, Homo sapiens
FWLEERDFEAGVFELEAIVNSIKRS

SEQ ID NO: 12 - protein, 40 aa, Homo sapiens
MKWDTDNTLGTEISWENKLAEGLKLTLDTIFVHHVLHAPH

SEQ ID NO: 13 - protein, 31 aa, Homo sapiens
RGAVFSQDKDVVQEATKVLRNAADNFYINDR

SEQ ID NO: 14 - protein, 33 aa, Homo sapiens
TGTGAPRFIKEVQELNSALHQSDLIDIYRTLHP

SEQ ID NO: 15 - protein, 20 aa, Homo sapiens
SNELTRAVEELHKLLKEARE

SEQ ID NO: 16 - protein, 33 aa, Homo sapiens
TYWNLLPPKRPIKEVLTDIFAKVLEKGWVDSRS

SEQ ID NO: 17 - protein, 19 aa, Homo sapiens
LFTILLTLWTMRCSSTPSG

SEQ ID NO: 18 - protein, 28 aa, Homo sapiens
AGEDMEISVKELRTILNRIISKHKDLRT

SEQ ID NO: 19 - protein, 26 aa, Homo sapiens
GLREESEEYMAAADEYNRLKQVKQPA

SEQ ID NO: 20 - protein, 67 aa, Homo sapiens
  VARIANT 58, 62, 65: Xaa = Any Amino Acid
KGIISRLMSVEEELKRDHAEMQAGCGLQTEDHLMPRRSAFASLDAVNARLMSALTPAXRYVXHCXPL

SEQ ID NO: 21 - protein, 26 aa, Homo sapiens
WERIEERLAYIADHLGFSWTELARAL

SEQ ID NO: 22 - protein, 27 aa, Homo sapiens
ARGDFAQAAQQLWLALRALGRPLPTSH

SEQ ID NO: 23 - protein, 30 aa, Homo sapiens
GSSKDLAKHIQVVCDGMDLTPKIHDLKPQC

SEQ ID NO: 24 - protein, 33 aa, Homo sapiens
GFLAAEQDIREEIRKVVQSLEQTAREVLTLLQG

SEQ ID NO: 25 - protein, 33 aa, Homo sapiens
LDPVKDVLILSALRRMLWAADDFLEDLPFEQIG

SEQ ID NO: 26 - protein, 31 aa, Homo sapiens
ANLLLLMVPILIAMAFLMLTERKILGYIQPR

SEQ ID NO: 27 - protein, 30 aa, Homo sapiens
LRLNTTVWPTIITPILLTLFLITNRLITTR

SEQ ID NO: 28 - protein, 26 aa, Homo sapiens
TLYLKLTALAVTFLGLLTALDLNYPT

SEQ ID NO: 29 - protein, 44 aa, Homo sapiens
AGVFSAEPSPFPQTRRSMVFARHLREVGDEFRSRHLNSTDDADE

SEQ ID NO: 30 - protein, 45 aa, Homo sapiens
GLKLATVAASMDRVPKVTPSSAISSIARENHEPERLGLNGIAETT

SEQ ID NO: 31 - protein, 26 aa, Homo sapiens
MRDLPGHYYETLKFLVGHLKTIADHR

SEQ ID NO: 32 - protein, 42 aa, Homo sapiens
CGGRMEDIPCSRVGHIYRKYVPYKVPAGVSLARNLKRVADWM

SEQ ID NO: 33 - protein, 37 aa, Homo sapiens
ALSWIEMDTEMEMLLARFRRTPGDLHLDHSVHLCAHP

SEQ ID NO: 34 - protein, 11 aa, Homo sapiens
TSTLPHIRRTR

SEQ ID NO: 35 - protein, 12 aa, Homo sapiens
NGNLFASFIADS

SEQ ID NO: 36 - protein, 29 aa, Homo sapiens
ILTSPWTTSSGLWPRLQKAAEAFKQLNQP

SEQ ID NO: 37 - protein, 32 aa, Homo sapiens
RTLQPRLLQNQQQHLPALPIWFLLQWLRLHPL

SEQ ID NO: 38 - protein, 37 aa, Homo sapiens
MAVIINELSQRDSCGPLKISLNNKILVYGNLFSSFTP

SEQ ID NO: 39 - protein, 16 aa, Homo sapiens
GLAKKSKRNPANLTPP

SEQ ID NO: 40 - protein, 20 aa, Homo sapiens
SSQALRIHQWLHLFSDFTST

SEQ ID NO: 41 - protein, 18 aa, Homo sapiens
GQVGRQLAIIGDDINRRK

SEQ ID NO: 42 - protein, 26 aa, Homo sapiens
GVSEAEGTFPLSTFLLGIASRLRSVA

SEQ ID NO: 43 - protein, 31 aa, Homo sapiens
RAPRFIKQILLDLKREIDFNVRLVEYFNPLS

SEQ ID NO: 44 - protein, 26 aa, Homo sapiens
IVAIIAGRLRMLGDQFNGELEASAKN

SEQ ID NO: 45 - protein, 29 aa, Homo sapiens
LALAYYSSRQYASALKHIAEIIERGIRQH

SEQ ID NO: 46 - protein, 38 aa, Homo sapiens
AAMLLDRRGTECDLWINEMSLLHKIVQDVYGTPHPPHS

SEQ ID NO: 47 - protein, 22 aa, Homo sapiens
PWQYKPIADLYRGRESRPSAPR

SEQ ID NO: 48 - protein, 18 aa, Homo sapiens
LFSVLLRYLADNFLPGGS

SEQ ID NO: 49 - protein, 18 aa, Homo sapiens
DWQVLLGKLLWKIDNPGI

SEQ ID NO: 50 - protein, 22 aa, Homo sapiens
GAMEREWAMFLRAASSRIRGGV

SEQ ID NO: 51 - protein, 24 aa, Homo sapiens
VHNFGRHWGLPLSFLLNYPLFLSP

SEQ ID NO: 52 - protein, 40 aa, Homo sapiens
ASMAPVGRDAETLQKQKETIKAFLKKLEALMASNDNANKT

SEQ ID NO: 53 - protein, 33 aa, Homo sapiens
CREQAELTGLRLASLGLKFNKIVHSSMTRAIET

SEQ ID NO: 54 - protein, 22 aa, Homo sapiens
GTRISDMLKLIADTWQRNCCPA

SEQ ID NO: 55 - protein, 26 aa, Homo sapiens
EQASVKYVILDMYRALLTLMNTSTAT

SEQ ID NO: 56 - protein, 20 aa, Homo sapiens
EDLESVLIRLINWAKGSPIP

SEQ ID NO: 57 - protein, 25 aa, Homo sapiens
RPVSFCGAVWTLNRAIGRHFVRGSR

SEQ ID NO: 58 - protein, 29 aa, Homo sapiens
HAVVARLLHIGAIMFQRLDFIEQLSAPPA

SEQ ID NO: 59 - protein, 31 aa, Homo sapiens
GQGTLWGSGMEAWLATVLKALPWHPTYQLEP

SEQ ID NO: 60 - protein, 28 aa, Homo sapiens [sequence omitted]

SEQ ID NO: 61 - protein, 22 aa, Homo sapiens [sequence omitted]

SEQ ID NO: 62 - protein, 39 aa, Homo sapiens
QRRGNEFQLRDLADAWDLSSRSRQRGWQMPNCRSRRGPG

SEQ ID NO: 63 - protein, 18 aa, Homo sapiens
RGLWVDRVLEEWGLEPRQ

SEQ ID NO: 64 - protein, 28 aa, Homo sapiens
FVRSVGWRLQNIGDDMDHAICGHDVRLG

SEQ ID NO: 65 - protein, 13 aa, Homo sapiens
SGLRKPTCGSSQR

SEQ ID NO: 66 - protein, 25 aa, Homo sapiens
AGTQPLILAQFMRVGGDELLHFLLW

SEQ ID NO: 67 - protein, 32 aa, Homo sapiens
MDTIKGFDLITNFQVVADALNISLLPNPLATA

SEQ ID NO: 68 - protein, 22 aa, Homo sapiens
ATWMKTLQGLLDRIQAFPSSPH

SEQ ID NO: 69 - protein, 30 aa, Homo sapiens
EANRKQPKPNNSSTAYYNFTGVSILPSYKP

SEQ ID NO: 70 - protein, 16 aa, Homo sapiens
GSLTHHINNIKPSSTR

SEQ ID NO: 71 - protein, 28 aa, Homo sapiens
VSCWPSYLKYPLSTASASLLATQLKSIA

SEQ ID NO: 72 - DNA, 199 bp, Homo sapiens
taatacgactcactatagggacaattactatttacaattcttacttcacaatggcttccatgaggcaggctgaacctgcagatatgcgcccagagatatggatcgcccaagagttgcggcgtattggagacgagtttaacgcctactatgcaagggaggattacaaagacgatgacgataaggcatccgctatttaaaa

SEQ ID NO: 73 - DNA, 126 bp, Homo sapiens
tactatttacaattctcctaacacaatgggggcaggtggggacggcagctcgccatcatcggggacgacatcaaccgacggaaagattacaaagacgatgacgataaggcatccgctattaaaaaa

SEQ ID NO: 74 - DNA, 160 bp, Homo sapiens
tttacaattctcctaacacaatgaagctgagcgagtgtctcaagcgcatcggggacgaactggacagtaacatggagctgcagaggatgattgccgccgtggacacagactccccccgagattacaaagacgatgacgataaggcatccgctattaaaaa

SEQ ID NO: 75 - DNA, 232 bp, Homo sapiens
taatacgactcactatagggacaattactatttacaattctttctctacaatgacagggaaggaagccatactgcggaggctggtggccctgctggaggaggaggcagaagtcattaaccagaagctggcctcggaccccgccctgcgcagcaagctggtccgcctgtcctccgactctttcgcccacctggattacaaagacgatgacgataaggcatccgctatttaaaa

SEQ ID NO: 76 - DNA, 172 bp, Homo sapiens
gactcactatagggacaattactatttacaattcttacttccaacgagggatgctctactaccagacagagaaatatgatttggctatcaaagaccttaaagaagccttgattcagcttcgagggaacaatgattacaaagacgatgacgataaggcatccgctatttaaaa

SEQ ID NO: 77 - DNA, 208 bp, Homo sapiens
taatacgactcactatagggacaattactatttacaattctcctaacacaatgggtggggaaagtgatactgacccccacttccaggatgcgctaatgcagctcgccaaagctgtggcaagtgctgcagctgccctggtcctcaaggccaagagtgtggcccaacgagattacaaagacgatgacgatagggcatccgctatttaaaa

SEQ ID NO: 78 - DNA, 199 bp, Homo sapiens
taatacgactcactatagggacaattactatttacaattctttctctacaatgggaacacgccaagacagaatgtttgagacaatggcgattgagattgaacaacttttggcaaggcttacaggggtaaatgataaaatggcagaatataccaacgctgattacaaagacgatgacgataaggcatccgctatttaaaa

SEQ ID NO: 79 - DNA, 181 bp, Homo sapiens
ctatttacaattctcctaacacaatggcggtacaggaggatccggtgcagcgggagattcaccaggactgggctaaccgggagtacattgagataatcaccagcagcatcaagaaaatcgcagactttctcaactcgttcgattacaaagacgatgacgataaggcatccgctattaaaaa

SEQ ID NO: 80 - DNA, 208 bp, Homo sapiens
taatacgactcactatagggacaattactatttacaattctcctaacacaatggcgactcgacaggccttaaatgagatctcggcccggcacagtgggatccagcagcttgaacgcagtattcgtgagctgcacgacatattcacttttctggctaccgaagtgcgagattacaaagacgatgacgataaggcatccgctatttaaaa

SEQ ID NO: 81 - DNA, 178 bp, Homo sapiens
taatacgactcactatagggacaattactatttacaattctttctctacaatgatgttctccgacatctacgggatccgggagatcgcggacgggttgtgcctggaggtggaggggaagatggtcagtaggccagaggattacaaagacgatgacgataaggcatccgctatttaaaa

SEQ ID NO: 82 - DNA, 169 bp, Homo sapiens
taatacgactcactatagggacaattactatttacaattctcctaacacaatgttttggctggaagaaagggactttgaggcgggtgtttttgaactagaagcaattgttaacagcatcaaaagaagcgattacaaagacgatgacgataaggcatccgctatttaaaa

SEQ ID NO: 83 - DNA, 214 bp, Homo sapiens
taatacgactcactatagggacaattactatttacaattcttacttcaatacaatgaaatgggacacagacaatactctagggacagaaatctcttgggagaataagttggctgaagggttgaaactgactcttgataccatatttgtacatcacgtcctgcatgccccacacgattacaaagacgatgacgataaggcatccgctatttaaaa

SEQ ID NO: 84 - DNA, 187 bp, Homo sapiens
taatacgactcactatagggacaattactatttacaattctttctctacaatgcggggggcagtgttctcccaggataaggacgtcgtgcaggaggccacaaaggtgctgaggaatgctgccgacaacttctacatcaacgacagggattacaaagacgatgacgataaggcatccgctatttaaaa

SEQ ID NO: 85 - DNA, 190 bp, Homo sapiens
gactcactatagggacaattactatttacaattctcctaacacaatgaccggtacaggagcacccagattcataaaggaagtccaggaattgaactcagctctacatcaatcggacctaatagacatctacagaactctccaccccgctgattacaaagacgatgacgataaggcatccgctatttaaaa

SEQ ID NO: 86 - DNA, 130 bp, Homo sapiens
tttacaattctcctaacacaatgacaaagagcaatgaactaacccgggcagtagaggaactacacaaacttttgaaagaagctagggaagattacaaagacgatgacgataaggcatccgctatttaaaa

SEQ ID NO: 87 - DNA, 199 bp, Homo sapiens
taatacgactcactatagggacaattactatttacaattctcctaacacaatgacctactggaacctgctgccccccaagcggcccatcaaagaggtgctgacggacatctttgccaaggtgctggagaagggctgggtggacagccgctccatccacgattacaaagacgatgacgataaggcatccgctatttaaaa

SEQ ID NO: 88 - DNA, 97 bp, Homo sapiens
ctatttacaattctcctaacactatggactatgagatgctcttcaactccttcagggattacaaagacgatgacgataaggcatccgctattaaaaa

SEQ ID NO: 89 - DNA, 178 bp, Homo sapiens
taatacgactcactatagggacaattactatttacaattctttctctacaatggccggggaggacatggagatcagcgtgaaggagttgcggacaatcctcaataggatcatcagcaaacacaaagacctgcggaccgattacaaagacgatgacgataaggcatccgctatttaaaa

SEQ ID NO: 90 - DNA, 172 bp, Homo sapiens
taatacgactcactatagggacaattactatttacaattctcctaacacaatgggactaagagaagaaagtgaagagtacatggctgctgctgatgaatacaatagactgaagcaagtgaagcaacctgcagattacaaagacgatgacgataaggcatccgctatttaaaa

SEQ ID NO: 91 - DNA, 318 bp, Homo sapiens
taatacgactcactatagggacaattactatttacaattctttctctacaatgaagggcatcatcagcaggttgatgtccgtggaggaagaactgaagagggaccacgcagagatgcaagcggctgtggactccaaacagaagatcattgatgcccaggagaagcgcattgcctcgttggatgccgccaatgcccgcctcatgagtgccctgacccagctgaaagagaggtacagcatgcaagcccgtaacggcatctcccccaccaaccccgcggattacaaagacgatgacgataaggcatccactatttaaaaaa

SEQ ID NO: 92 - DNA, 172 bp, Homo sapiens
taatacgactcactatagggacaaatactatttacaattctcctaacacaatgtgggaacggattgaggaaaggctggcttatattgctgatcaccttggcttcagctggacagaattagcaagagcgctggattacaaagacgatgacgataaggcatccgctatttaaaa

SEQ ID NO: 93 - DNA, 177 bp, Homo sapiens
taatacgactcactataggggacaattactatttacaattgcttacttcacaatggctcggggagactttgcccaggctgcccagcagctgtggctggccctgcgggcactgggccggcccctgcccacctcccacgattacaaagacgatgacgataaggcatccgctatttaaaa

SEQ ID NO: 94 - DNA, 160 bp, Homo sapiens
taatacgactcactatagggacaattactatttacaattctttctctacaatggtggtggatgtgccagattttatagtctggcttgaggaggcagtatctgatttacatagggccctcgattacaaagacgatgacgataaggcatccgctatttaaaa

SEQ ID NO: 95 - DNA, 170 bp, Homo sapiens
  misc_feature 167: n = A,T,C or G
cttttacaattctcctaacacaatgggctttttggctgccgagcaggacatccgagaggaaatcagaaaagttgtacagagtttagaacaaacagctcgagaggttttaactctactgcaaggggtccaggattacaaagacgatgacgataaggcatccgctaagnaaa

SEQ ID NO: 96 - DNA, 227 bp, Homo sapiens
ttaatacgactcactatagggattactatttacaattcttacttcacaatgctggaccctgtaaaggatgttctaattctttctgctctgagacgaatgctatgggctgcagatgacttcttagaggatttgccttttgagcaaatagggaatctaagggaggaaattatcaactgtgcacaagcggattacaaagacgatgacgataaggcatccgctatttaaaa

SEQ ID NO: 97 - DNA, 161 bp, Homo sapiens
  misc_feature 158: n = A,T,C or G
ttctatttacaattctcctaacacaatggccaacctcctactcctcatggtacccattctaatcgcaatggcattcctaatgcttaccgaacgaaaaattctaggctatatacaaccacgcgattacaaagacgatgacgataaggcatccgctaaanaaa

SEQ ID NO: 98 - DNA, 149 bp, Homo sapiens
  misc_feature 16: n = A,T,C or G
aattctcctaacacantgctccggctaaatactaccgtatggcccaccataattacccccatactccttacactattcctcatcaccaaccgactaatcaccacccgggattacaaagacgatgacgataaggcatccgctatttaaaa

SEQ ID NO: 99 - DNA, 146 bp, Homo sapiens
  misc_feature 140: n = A,T,C or G
ctatttacaattctcctaacacaatgaccctctacctaaaactcacagccctcgctgtcactttcctaggacttctaacagccctagacctcaactacccaaccgattacaaagacgatgacgataaggcatccgctatnaaaaaa

SEQ ID NO: 100 - DNA, 226 bp, Homo sapiens
taatacgactcactatagggacaattactatttacaattctcctaacacaatggcgggcgtgttctcagccgagccgtcgccgtttccacagacccgtcgcagcatggtgtttgccaggcacctgcgggaggtgggagacgagttcaggagcagacatctcaactccacggacgacgcagacgaggattacaaagacgatgacgataaggcatccgctatttaaaa

SEQ ID NO: 101 - DNA, 229 bp, Homo sapiens
taatacgactcactatagggacaattactatttacaattctttctctacaatgggcttaaaacttgccacagttgctgccagtatggacagagtgccaaaggttactcccagcagtgccatcagcagcatagcaagagagaaccacgaaccagaaagattgggcttaaatggaatagcagagacaacagattacaaagacgatgacgataaggcatccgctatttaaaa

SEQ ID NO: 102 - DNA, 172 bp, Homo sapiens
taatacgactcactatagggacaattactatttacaattctcctaacacaatgatgcgggatctcccaggacactactatgaaacgctcaaattccttgtgggccatctcaagaccatcgctgaccaccgcgattacaaagacgatgacgataaggcatccgctatttaaaa

SEQ ID NO: 103 - DNA, 225 bp, Homo sapiens
taatacgactcactatagggacaattactatttacaattctttctctaggtgtggatgtgtgggggccgcatggaggacatcccctgctccagggtgggccatatctacaggaagtatgtgccctacaaggtcccggccggagtcagcctggcccggaaccttaagcgggtggccgattggatggattacaaagacgatgacgataaggcatccgctatttaaaa

SEQ ID NO: 104 - DNA, 205 bp, Homo sapiens
taatacgactcactatagggacaattactatttacaattctttctctacaatggcgcttagttggatcgaaatggacaccgagatggagatgcttctggctagatttcgcagaaccccaggagacctgcatttagaccactctgtccatttgtgtgcccaccccgattacaaagacgatgacgataaggcatccgctatttaaaa

SEQ ID NO: 105 - DNA, 101 bp, Homo sapiens
ctatttacaattctcctaacacaatgacctccaccctaccacacattcgaagaacccgtgattacaaagacgatgacgataaggcatccgctatttaaaaa

SEQ ID NO: 106 - DNA, 130 bp, Homo sapiens
taatacgactcactatagggacaattactatttacaattctcctaacacaatgaacggaaatctgttcgcttcattcatcgccgacagtgattacaaagacgatgacgataaggcatccgctatttaaaa

SEQ ID NO: 107 - DNA, 164 bp, Homo sapiens
taatacgactcactatagggacaattactatttacaattcttacttcgccctggacgacatcgagtggtttgtggccccggctgcagaaggcagccgaggctttcaagcagctgaaccagcccgattacaaagaccatgacgataaggcatccgctatttaaaa

SEQ ID NO: 108 - DNA, 192 bp, Homo sapiens
taatacgactcctatagggacaattactatttacaattcttacttcaatacaatgcgcaccctgcaacccaggcttcttcaaaaccaacaacagcacctgccagccctgcccatatggttcctactccaatggctcagactgcacccgctggattacaaagacgatgacgataaggcatccgctatttaaaa

SEQ ID NO: 109 - DNA, 210 bp, Homo sapiens
taatacgactcactatagggacaattactatttacaattctcctaacgccaaagcacaatggctgttataattaacgaattatctcagcgtgacagctgtggtcctttgaaaattagcttgaataacaagatcctggtgtatggtaatttattttcctctttcacccccgattacaaagacgatgacgataaggcatccgctatttaaaa

SEQ ID NO: 110 - DNA, 109 bp, Homo sapiens
caattctcctaacacgatgggactggctaaaaaaagtaaaaggaacccggcaaatcttaccccgcctgattacaaagacgatgacgataaggcatccgctatttaaaaa

SEQ ID NO: 111 - DNA, 131 bp, Homo sapiens
  misc_feature 1, 125, 126: n = A,T,C or G
natttctatttacaattctcctaacacaatgagctcacaggcacttagaatccatcagtggctccatcttttctcagacttcacctccaccgattacaaagacgatgacgataaggcatccgctnnaaaaa

SEQ ID NO: 112 - DNA, 172 bp, Homo sapiens
taatacgactcactatagggacaattactatttacaattctttctctacaatggaccaacccataggaaaatgggaaaagttgttcccgttacaactttacaaaacgttacaaatgctcatgtcccagatggattacaaagacgatgacgataaggcatccgctatttaaaa

SEQ ID NO: 113 - DNA, 172 bp, Homo sapiens
taatacgactcactatagggacaattactatttacaattcttacttcacaatgggggtctctgaggccgagggaacattcccgctcagcactttccttcttgggatagcatcccgtctaagaagcgtggctgattacaaagacgatgacgataaggcatccgctatttaaaa

SEQ ID NO: 114 - DNA, 187 bp, Homo sapiens
taatacgactcactatagggacaattactatttacaattctcctaacacaatgagggcgcccagattcataaagcaaatattgctagatctaaagagagagatagacttcaatgtgagattagtagaatacttcaacccactatcagattacaaagacgatgacgataaggcatccgctatttaaaa

SEQ ID NO: 115 - DNA, 172 bp, Homo sapiens
taatacgactcactatagggacaattactatttacaattctttctctacaatgatcgtggctatcattgctggtcgccttcggatgttgggtgaccagttcaacggagaattggaagcttctgccaaaaacgattacaaagacgatgacgataaggcatccgctatttaaaa

SEQ ID NO: 116 - DNA, 180 bp, Homo sapiens
taatacgactcactatagggacaattactatttacaattctttctctacaacctggctttggcctattacagcagccgacagtatgcttcagcactgaagcatatcgctgagattattgagcgtggcatccgccagcacgattacaaagacgatgacgataaggcatccgctatttaaaa

SEQ ID NO: 117 - DNA, 208 bp, Homo sapiens
taatacgactcactatagggacaattactatttacaattctttctctacgatggctgccatgttattagacagaagaggaactgagtgtgacctctggataaatgagatgtcactattacataagattgttcaagatgtatatggaactcctcacccgccccactccgattacaaagacgatgacgataaggcatccgctatttaaaa

SEQ ID NO: 118 - DNA, 160 bp, Homo sapiens
taatacgactcactatagggacaattactatttacaattctcctaacacaatgccttggcaatacaaaccgatagctgatctttacagagggagagagagccgtccctctgccccccgggattacaaagacgatgacgataaggcatccgctatttaaaa

SEQ ID NO: 119 - DNA, 148 bp, Homo sapiens
taatacgactcactatagggacaattactatttacaattctttctctacaatgctgttctcagtgttgctacgttatttggcagataactttctgccaggaggatccgattacaaagacgatgacgataaggcatccgctatttaaaa

SEQ ID NO: 120 - DNA, 147 bp, Homo sapiens
taatacgactcactatagggacaattactatttacaattctcctaacacaatggattggcaggtgttgctaggaaaactactttggaaaatagataatccgggcatcgattacaaagacgatgacgataggcatccgctatttaaaa

SEQ ID NO: 121 - DNA, 160 bp, Homo sapiens
taatacgactcactatagggacaattactatttacaattctttctctacaatgggtgctatggagagagaatgggcgatgtttctcagggctgcttcaagcaggattaggggtggcgtggattacaaagacgatgacgataaggcatccgctgtttaaaa

SEQ ID NO: 122 - DNA, 140 bp, Homo sapiens
ctatttacaattctcctaacacaatggtgcataactttgggagacactggggtctgcccttgagttttcttctcaattaccctttattcctcagtccggattacaaagacgatgacgataaggcatccgctattaaaaaa

SEQ ID NO: 123 - DNA, 211 bp, Homo sapiens
taatacgactcactataggaaatactatttacaattcttacttcacaatggctagcatggctccagtggggagagatgcagaaacattgcaaaagcaaaaggaaactataaaagcctttctaaagaaactagaagccctcatggcaagcaatgacaatgccaataaaaccgatgacaaagacgatgacgataaggcatccgctatttaaaa

SEQ ID NO: 124 - DNA, 196 bp, Homo sapiens
taatacgactcactatagggacaattactatttacaattctttctctacaatgtgtcgggagcaggctgaactcactgggctccgcctggcaagcttggggttgaagtttaataaaatcgtccattcgtctatgacgcgcgccatagagaccaccgattacaaagacgatgacgataaggcatccgctatttaaaa

SEQ ID NO: 125 - DNA, 161 bp, Homo sapiens
taatacgactcactataggggacaattactatttacaattcttacttcacaatgggcactagaattagtgatatgctaaaattaattgcagacacatggcagagaaattgttgccctgcggattacaaagacgatgacgataaggcatccgctatttaaaa

SEQ ID NO: 126 - DNA, 172 bp, Homo sapiens
taatacgactcactatagggacaattactatttacaattctcctaacacaatggagcaggccagtgttaagtatgttattctggatatgtacagagcactcttgacactaatgaatacttcaacagccacagattacaaagacgatgacgataaggcatccgctatttaaaa

SEQ ID NO: 127 - DNA, 120 bp, Homo sapiens
caattctcctaacacaatggaagacctagagagtgtgttaataagactgatcaactgggcaaaaggaagccccatcccagattacaaagacgatgacgataaggcatccgctatttaaaa

SEQ ID NO: 128 - DNA, 169 bp, Homo sapiens
taatacgactcactatagggacaattactatttacaattctcctaacacaatgaggccggtgtccttttgcggggctgtttggactctgaacagggcaataggaaggcattttgtccgaggtagcagggattacaaagacgatgacgataaggcatccgctatttaaaa

SEQ ID NO: 129 - DNA, 181 bp, Homo sapiens
taatacgactcactatagggacaattactatttacaattctttctctacaatgcacgcggtggtggcacgtttgcttcacattggggcaatcatgttccaacgactagacttcatagaacaattgtctgcacccccagcggattacaaagacgatgacgataaggcatccgctatttaaaa

SEQ ID NO: 130 - DNA, 159 bp, Homo sapiens
  misc_feature 155: n = A,T,C or G
cttttacaattctcctaacacaatgggccaaggtacactttggggaagtgggatggaagcatggttggcaacggtgttgaaggcactcccttggcaccccacataccagctggagccggattacaaagacgatgacgataaggcatccgctatanaaaa

SEQ ID NO: 131 - DNA, 148 bp, Homo sapiens
  misc_feature 147: n = A,T,C or G
ttctatttacaattctcctaacacaatgatagcacaggcaacgaaagcaacaatagacaaatggaactgcatcaaacttaaaatcttctacacctcaaagaaagaagccagcgattacaaagacgatgacgataaggcatccgctant

SEQ ID NO: 132 - DNA, 160 bp, Homo sapiens
taatacgactcactatagggacaattactatttacaattctttctctacaatggtggtggatgtgccagattttatagtctggcttgaggaggcagtatctgatttacatagagccctcgattacaaagacgatgacgataaggcatccgctatttaaaa

SEQ ID NO: 133 - DNA, 211 bp, Homo sapiens
taatacgactcactatagggacaattactatttacaattctttctctacaatgcagaggagagggaatgaattccagctgagagacctggccgatgcatgggatttgtcttcaaggtccaggcagaggggatggcagatgccaaattgcagaagtcgaagagggcccggagattacaaagacgatgacgataaggcatccgctatttaaaa

SEQ ID NO: 134 - DNA, 118 bp, Homo sapiens
tttacaattctcctaacacaatgcggggcctgtgggtggacagggtcctagaggaatggggcctggaaccgcggcaggattacaaagacgatgacgataaggcatccgctattaaaaa

SEQ ID NO: 135 - DNA, 179 bp, Homo sapiens
taatacgactcactatagggacaattactatttacaattctttactctacaatgttcgtgaggtctgttggctggaggctgcagaacattggtgatgacatggaccacgccatttgtggccatgatgtcaggctcggcgattacaaagacgatgacgataaggcatccgctatttaaaa

SEQ ID NO: 136 - DNA, 82 bp, Homo sapiens
gcagtggactcagaaagccaacatgtggctcctcccagcgcgattacaaagacgatgacgataaggcatccgctatttaaaa

SEQ ID NO: 137 - DNA, 169 bp, Homo sapiens
taatacgactcactatagggacaattactatttacaattctttctctacaatggcgggtacacagccacttatccttgcccagttcatgcgtgttggaggtgacgaacttctccacttcctgctctgggattacaaagacgatgacgataaggcatccgctatttaaaa

SEQ ID NO: 138 - DNA, 190 bp, Homo sapiens
taatacgactcactatagggacaattactatttacaattctcctaacaccatgatggataccataaagggatttgacctaatcactaattttcaggtggtggctgatgctttgaacatctctttgctgcccaatccattagcgacagcggattacaaagacgatgacgataaggcatacgctatttaaaa

SEQ ID NO: 139 - DNA, 135 bp, Homo sapiens
  misc_feature 128: n = A,T,C or G
tctatttacaattctcctaacacaatggccacttggatgaaaacccttcaaggattactggatagaattcaggctttcccctccagcccccacgattacaaagacgatgacgataaggcatccgctangaaaaaa

SEQ ID NO: 140 - DNA, 159 bp, Homo sapiens
ctatttacaattctcctaacacaatggaagctaatagaaaacaaccgaaaccaaataattcaagcactgcttattacaattttactggggtctctattttaccctcctacaagccccagattacaaagacgatgacgataaggcatccgctataaaaaa

SEQ ID NO: 141 - DNA, 118 bp, Homo sapiens
  misc_feature 112: n = A,T,C or G
ttctatttacaattctcctaacacaatggggctcactcacccaccacattaacaacataaaaccctcatccacacgagattacaaagacgatgacgataaggcatccgctanaaaaaa

SEQ ID NO: 142 - DNA, 177 bp, Homo sapiens
taatacgactcatatagggacaattactatttacaattcttacttcacaatggtgagctgctggccgattactaaaataccctttgtctacagcctccgcttctctcctggctacgcaattgaaaagcatagcggattacaaagacgatgacgataaggcatccgctatttaaaaaa

SEQ ID NO: 143 - DNA, 71 bp, Artificial Sequence
  Oligonucleotide Primer
taatacgactcactatagggacaattactatttacaatthhhhhhhhacaatggctgaagaacagaaactg

SEQ ID NO: 144 - DNA, 39 bp, Artificial Sequence
  Oligonucleotide Primer
taatacgactcactatagggacaattactatttacaatt

SEQ ID NO: 145 - DNA, 33 bp, Artificial Sequence
  misc_feature 25, 26, 27, 28, 29, 30, 31, 32, 33: n = A,T,C or G
ggaacttgcttcgtctttgcaatcnnnnnnnnn

SEQ ID NO: 146 - DNA, 33 bp, Artificial Sequence
  misc_feature 25, 26, 27, 28, 29, 30, 31, 32, 33: n = A,T,C or G
ggatgatgcttcgtctttgtaatcnnnnnnnnn

SEQ ID NO: 147 - DNA, 45 bp, Artificial Sequence
  misc_feature 36, 37, 38, 39, 40, 41, 42, 43, 44, 45: n = A,T,C or G
ggacaattactatttacaatthhhhhhhhacaatgnnnnnnnnnn

SEQ ID NO: 148 - DNA, 39 bp, Artificial Sequence; identical to SEQ ID NO: 144
  Oligonucleotide Primer

SEQ ID NO: 149 - DNA, 41 bp, Artificial Sequence
  Oligonucleotide Primer
ttttaaatagcgcatgccttatcgtcatcgtctttgtaatc

SEQ ID NO: 150 - DNA, 30 bp, Artificial Sequence
  Oligonucleotide Primer
agtatcgaattcatgtctcagagcaaccgg

SEQ ID NO: 151 - DNA, 35 bp, Artificial Sequence
  Oligonucleotide Primer
tacagtctcgagctagttgaagcgttcctggccct

SEQ ID NO: 152 - protein, 28 aa, Homo sapiens
MGQVGRQLAIIGDDINRDYKDDDDKASA

SEQ ID NO: 153 - DNA, 105 bp, Homo sapiens
gcttccatgaggcaggctgaacctgcagatatgcgcccagagatatggatcgcccaagagttgcggcgtattggagacgagtttaacgcctactatgcaagggag

SEQ ID NO: 154 - DNA, 56 bp, Homo sapiens
ggggcaggtggggacggcagctcgccatcatcggggacgacatcaaccgacggaaa

SEQ ID NO: 155 - DNA, 96 bp, Homo sapiens
aagctgagcgagtgtctcaagcgcatcggggacgaactggacagtaacatggagctgcagaggatgattgccgccgtggacacagactccccccga

SEQ ID NO: 156 - DNA, 138 bp, Homo sapiens
acagggaaggaagccatactgcggaggctggtggccctgctggaggaggaggcagaagtcattaaccagaagctggcctcggaccccgccctgcgcagcaagctggtccgcctgtcctccgactctttcgcccacctg

SEQ ID NO: 157 - DNA, 78 bp, Homo sapiens
ctctactaccagacagagaaatatgatttggctatcaaagaccttaaagaagccttgattcagcttcgagggaacaat

SEQ ID NO: 158 - DNA, 114 bp, Homo sapiens
ggtggggaaagtgatactgacccccacttccaggatgcgctaatgcagctcgccaaagctgtggcaagtgctgcagctgccctggtcctcaaggccaagagtgtggcccaacga

SEQ ID NO: 159 - DNA, 105 bp, Homo sapiens
ggaacacgccaagacagaatgtttgagacaatggcgattgagattgaacaacttttggcaaggcttacaggggtaaatgataaaatggcagaatataccaacgct

SEQ ID NO: 160 - DNA, 114 bp, Homo sapiens
gcggtacaggaggatccggtgcagcgggagattcaccaggactgggctaaccgggagtacattgagataatcaccagcagcatcaagaaaatcgcagactttctcaactcgttc

SEQ ID NO: 161 - DNA, 114 bp, Homo sapiens
gcgactcgacaggccttaaatgagatctcggcccggcacagtgggatccagcagcttgaacgcagtattcgtgagctgcacgacatattcacttttctggctaccgaagtgcga

SEQ ID NO: 162 - DNA, 84 bp, Homo sapiens
atgttctccgacatctacgggatccgggagatcgcggacgggttgtgcctggaggtggaggggaagatggtcagtaggccagag

SEQ ID NO: 163 - DNA, 75 bp, Homo sapiens
ttttggctggaagaaagggactttgaggcgggtgtttttgaactagaagcaattgttaacagcatcaaaagaagc

SEQ ID NO: 164 - DNA, 117 bp, Homo sapiens
aaatgggacacagacaatactctagggacagaaatctcttgggagaataagttggctgaagggttgaaactgactcttgataccatatttgtacatcacgtcctgcatgccccacac

SEQ ID NO: 165 - DNA, 93 bp, Homo sapiens
cggggggcagtgttctcccaggataaggacgtcgtgcaggaggccacaaaggtgctgaggaatgctgccgacaacttctacatcaacgacagg

SEQ ID NO: 166 - DNA, 102 bp, Homo sapiens
accggtacaggagcacccagattcataaaggaagtccaggaattgaactcagctctacatcaatcggacctaatagacatctacagaactctccaccccgct

SEQ ID NO: 167 - DNA, 66 bp, Homo sapiens
acaaagagcaatgaactaacccgggcagtagaggaactacacaaacttttgaaagaagctagggaa

SEQ ID NO: 168 - DNA, 105 bp, Homo sapiens
acctactggaacctgctgccccccaagcggcccatcaaagaggtgctgacggacatctttgccaaggtgctggagaagggctgggtggacagccgctccatccac

SEQ ID NO: 169 - DNA, 30 bp, Homo sapiens
gactatgagatgctcttcaactccttcagg

SEQ ID NO: 170 - DNA, 84 bp, Homo sapiens
gccggggaggacatggagatcagcgtgaaggagttgcggacaatcctcaataggatcatcagcaaacacaaagacctgcggacc

SEQ ID NO: 171 - DNA, 78 bp, Homo sapiens
ggactaagagaagaaagtgaagagtacatggctgctgctgatgaatacaatagactgaagcaagtgaagcaacctgca

SEQ ID NO: 172 - DNA, 222 bp, Homo sapiens
aagggcatcatcagcaggttgatgtccgtggaggaagaactgaagagggaccacgcagagatgcaagcggctgtggactccaaacagaagatcattgatgcccaggagaagcgcattgcctcgttggatgccgccaatgcccgcctcatgagtgccctgacccagctgaaagagaggtacagcatgcaagcccgtaacggcatctcccccaccaaccccgcg

SEQ ID NO: 173 - DNA, 78 bp, Homo sapiens
tgggaacggattgaggaaaggctggcttatattgctgatcaccttggcttcagctggacagaattagcaagagcgctg

SEQ ID NO: 174 - DNA, 81 bp, Homo sapiens
gctcggggagactttgcccaggctgcccagcagctgtggctggccctgcgggcactgggccggcccctgcccacctcccac

SEQ ID NO: 175 - DNA, 66 bp, Homo sapiens
gtggtggatgtgccagattttatagtctggcttgaggaggcagtatctgatttacatagggccctc

SEQ ID NO: 176 - DNA, 105 bp, Homo sapiens
ggctttttggctgccgagcaggacatccgagaggaaatcagaaaagttgtacagagtttagaacaaacagctcgagaggttttaactctactgcaaggggtccag

SEQ ID NO: 177 - DNA, 135 bp, Homo sapiens
ctggaccctgtaaaggatgttctaattctttctgctctgagacgaatgctatgggctgcagatgacttcttagaggatttgccttttgagcaaatagggaatctaagggaggaaattatcaactgtgcacaagcg

SEQ ID NO: 178 - DNA, 93 bp, Homo sapiens
gccaacctcctactcctcatggtacccattctaatcgcaatggcattcctaatgcttaccgaacgaaaaattctaggctatatacaaccacgc

SEQ ID NO: 179 - DNA, 90 bp, Homo sapiens
ctccggctaaatactaccgtatggcccaccataattacccccatactccttacactattcctcatcaccaaccgactaatcaccacccgg

SEQ ID NO: 180 - DNA, 78 bp, Homo sapiens
accctctacctaaaactcacagccctcgctgtcactttcctaggacttctaacagccctagacctcaactacccaacc

SEQ ID NO: 181 - DNA, 132 bp, Homo sapiens
gcgggcgtgttctcagccgagccgtcgccgtttccacagacccgtcgcagcatggtgtttgccaggcacctgcgggaggtgggagacgagttcaggagcagacatctcaactccacggacgacgcagacgag

SEQ ID NO: 182 - DNA, 135 bp, Homo sapiens
ggcttaaaacttgccacagttgctgccagtatggacagagtgccaaaggttactcccagcagtgccatcagcagcatagcaagagagaaccacgaaccagaaagattgggcttaaatggaatagcagagacaaca

SEQ ID NO: 183 - DNA, 78 bp, Homo sapiens
atgcgggatctcccaggacactactatgaaacgctcaaattccttgtgggccatctcaagaccatcgctgaccaccgc

SEQ ID NO: 184 - DNA, 126 bp, Homo sapiens
tgtgggggccgcatggaggacatcccctgctccagggtgggccatatctacaggaagtatgtgccctacaaggtcccggccggagtcagcctggcccggaaccttaagcgggtggccgattggatg

SEQ ID NO: 185 - DNA, 111 bp, Homo sapiens
gcgcttagttggatcgaaatggacaccgagatggagatgcttctggctagatttcgcagaaccccaggagacctgcatttagaccactctgtccatttgtgtgcccacccc

SEQ ID NO: 186 - DNA, 33 bp, Homo sapiens
acctccaccctaccacacattcgaagaacccgt

SEQ ID NO: 187 - DNA, 36 bp, Homo sapiens
aacggaaatctgttcgcttcattcatcgccgacagt

SEQ ID NO: 188 - DNA, 70 bp, Homo sapiens
gacgacatcgagtggtttgtggccccggctgcagaaggcagccgaggctttcaagcagctgaaccagccc

SEQ ID NO: 189 - DNA, 96 bp, Homo sapiens
cgcaccctgcaacccaggcttcttcaaaaccaacaacagcacctgccagccctgcccatatggttcctactccaatggctcagactgcacccgctg

SEQ ID NO: 190 - DNA, 108 bp, Homo sapiens
gctgttataattaacgaattatctcagcgtgacagctgtggtcctttgaaaattagcttgaataacaagatcctggtgtatggtaatttattttcctctttcaccccc

SEQ ID NO: 191 - DNA, 48 bp, Homo sapiens
ggactggctaaaaaaagtaaaaggaacccggcaaatcttaccccgcct

SEQ ID NO: 192 - DNA, 60 bp, Homo sapiens
  misc_feature 1, 125, 126: n = A,T,C or G
agctcacaggcacttagaatccatcagtggctccatcttttctcagacttcacctccacc

SEQ ID NO: 193 - DNA, 78 bp, Homo sapiens
gaccaacccataggaaaatgggaaaagttgttcccgttacaactttacaaaacgttacaaatgctcatgtcccagatg

SEQ ID NO: 194 - DNA, 78 bp, Homo sapiens
ggggtctctgaggccgagggaacattcccgctcagcactttccttcttgggatagcatcccgtctaagaagcgtggct

SEQ ID NO: 195 - DNA, 93 bp, Homo sapiens
agggcgcccagattcataaagcaaatattgctagatctaaagagagagatagacttcaatgtgagattagtagaatacttcaacccactatca

SEQ ID NO: 196 - DNA, 78 bp, Homo sapiens
atcgtggctatcattgctggtcgccttcggatgttgggtgaccagttcaacggagaattggaagcttctgccaaaaac

SEQ ID NO: 197 - DNA, 84 bp, Homo sapiens
gctttggcctattacagcagccgacagtatgcttcagcactgaagcatatcgctgagattattgagcgtggcatccgccagcac

SEQ ID NO: 198 - DNA, 114 bp, Homo sapiens
gctgccatgttattagacagaagaggaactgagtgtgacctctggataaatgagatgtcactattacataagattgttcaagatgtatatggaactcctcacccgccccactcc

SEQ ID NO: 199 - DNA, 66 bp, Homo sapiens
ccttggcaatacaaaccgatagctgatctttacagagggagagagagccgtccctctgccccccgg

SEQ ID NO: 200 - DNA, 54 bp, Homo sapiens
ctgttctcagtgttgctacgttatttggcagataactttctgccaggaggatcc

SEQ ID NO: 201 - DNA, 54 bp, Homo sapiens
gattggcaggtgttgctaggaaaactactttggaaaatagataatccgggcatc

SEQ ID NO: 202 - DNA, 66 bp, Homo sapiens
ggtgctatggagagagaatgggcgatgtttctcagggctgcttcaagcaggattaggggtggcgtg

SEQ ID NO: 203 - DNA, 72 bp, Homo sapiens
gtgcataactttgggagacactggggtctgcccttgagttttcttctcaattaccctttattcctcagtccg

SEQ ID NO: 204 - DNA, 120 bp, Homo sapiens
gctagcatggctccagtggggagagatgcagaaacattgcaaaagcaaaaggaaactataaaagcctttctaaagaaactagaagccctcatggcaagcaatgacaatgccaataaaacc

SEQ ID NO: 205 - DNA, 102 bp, Homo sapiens
tgtcgggagcaggctgaactcactgggctccgcctggcaagcttggggttgaagtttaataaaatcgtccattcgtctatgacgcgcgccatagagaccacc

SEQ ID NO: 206 - DNA, 66 bp, Homo sapiens
ggcactagaattagtgatatgctaaaattaattgcagacacatggcagagaaattgttgccctgcg

SEQ ID NO: 207 - DNA, 78 bp, Homo sapiens
gagcaggccagtgttaagtatgttattctggatatgtacagagcactcttgacactaatgaatacttcaacagccaca

SEQ ID NO: 208 - DNA, 60 bp, Homo sapiens
gaagacctagagagtgtgttaataagactgatcaactgggcaaaaggaagccccatccca

SEQ ID NO: 209 - DNA, 75 bp, Homo sapiens
aggccggtgtccttttgcggggctgtttggactctgaacagggcaataggaaggcattttgtccgaggtagcagg

SEQ ID NO: 210 - DNA, 87 bp, Homo sapiens
cacgcggtggtggcacgtttgcttcacattggggcaatcatgttccaacgactagacttcatagaacaattgtctgcacccccagcg

SEQ ID NO: 211 - DNA, 93 bp, Homo sapiens
  misc_feature 155: n = A,T,C or G
ggccaaggtacactttggggaagtgggatggaagcatggttggcaacggtgttgaaggcactcccttggcaccccacataccagctggagccg

SEQ ID NO: 212 - DNA, 84 bp, Homo sapiens
  misc_feature 147: n = A,T,C or G
atagcacaggcaacgaaagcaacaatagacaaatggaactgcatcaaacttaaaatcttctacacctcaaagaaagaagccagc

SEQ ID NO: 213 - DNA, 66 bp, Homo sapiens
gtggtggatgtgccagattttatagtctggcttgaggaggcagtatctgatttacatagagccctc

SEQ ID NO: 214 - DNA, 117 bp, Homo sapiens
cagaggagagggaatgaattccagctgagagacctggccgatgcatgggatttgtcttcaaggtccaggcagaggggatggcagatgccaaattgcagaagtcgaagagggcccgga

SEQ ID NO: 215 - DNA, 54 bp, Homo sapiens
cggggcctgtgggtggacagggtcctagaggaatggggcctggaaccgcggcag

SEQ ID NO: 216 - DNA, 84 bp, Homo sapiens
ttcgtgaggtctgttggctggaggctgcagaacattggtgatgacatggaccacgccatttgtggccatgatgtcaggctcggc

SEQ ID NO: 217 - DNA, 39 bp, Homo sapiens
agtggactcagaaagccaacatgtggctcctcccagcgc

SEQ ID NO: 218 - DNA, 75 bp, Homo sapiens
gcgggtacacagccacttatccttgcccagttcatgcgtgttggaggtgacgaacttctccacttcctgctctgg

SEQ ID NO: 219 - DNA, 96 bp, Homo sapiens
atggataccataaagggatttgacctaatcactaattttcaggtggtggctgatgctttgaacatctctttgctgcccaatccattagcgacagcg

SEQ ID NO: 220 - DNA, 66 bp, Homo sapiens
  misc_feature 128: n = A,T,C or G
gccacttggatgaaaacccttcaaggattactggatagaattcaggctttcccctccagcccccac

SEQ ID NO: 221 - DNA, 92 bp, Homo sapiens
gaagctaatagaaaacaaccgaaaccaaataattcaagcactgcttattacaattttactggggtctctattttaccctcctacaagcccca

SEQ ID NO: 222 - DNA, 49 bp, Homo sapiens
  misc_feature 112: n = A,T,C or G
gggctcactcacccaccacattaacaacataaaaccctcatccacacga

SEQ ID NO: 223 - DNA, 82 bp, Homo sapiens
gtgagctgctggccgattactaaaataccctttgtctacagcctccgcttctctcctggctacgcaattgaaaagcatagcg

SEQ ID NO: 224 - protein, 11 aa, Homo sapiens
KYQQLFEDIRW

SEQ ID NO: 225 - protein, 16 aa, Homo sapiens
IGEEFSRAAEKLYLAV

SEQ ID NO: 226 - protein, 23 aa, Homo sapiens
KAEVQIARKLQCIADQFHRLHVL

SEQ ID NO: 227 - protein, 22 aa, Homo sapiens
MGDVVGFIDELEGAVSDLHRAL

SEQ ID NO: 228 - protein, 15 aa, Homo sapiens
TLRHWGLQFNTRFGV

SEQ ID NO: 229 - protein, 14 aa, Homo sapiens
SRREEAWDALFRGI

SEQ ID NO: 230 - protein, 17 aa, Homo sapiens
TLREIGDLYLTSILGRR

SEQ ID NO: 231 - DNA, 33 bp, Homo sapiens
aaataccagcaactttttgaagatattcggtgg

SEQ ID NO: 232 - DNA, 48 bp, Homo sapiens
atcggggaggagttcagccgcgctgccgagaagctttacctcgctgtt

SEQ ID NO: 233 - DNA, 69 bp, Homo sapiens
aaagcagaggtacagattgcccgaaagcttcagtgcattgcagaccagttccaccggcttcatgtgctt

SEQ ID NO: 234 - DNA, 66 bp, Homo sapiens
atgggagatgtggttggttttatagacgaacttgagggggcagtgtctgatttacatagggcgttg

SEQ ID NO: 235 - DNA, 45 bp, Homo sapiens
acactccgacactggggattacagttcaacacaagatttggtgtg

SEQ ID NO: 236 - DNA, 42 bp, Homo sapiens
tcgagaagggaagaggcatgggatgctttatttcgtgggatc

SEQ ID NO: 237 - DNA, 42 bp, Homo sapiens; identical to SEQ ID NO: 236

SEQ ID NO: 238 - protein, 18 aa, Homo sapiens
MPVVHLTLTTAGDDFSRR

SEQ ID NO: 239 - protein, 25 aa, Homo sapiens
MPQDASTKKLSECLKRIGDELDSNG

SEQ ID NO: 240 - protein, 17 aa, Homo sapiens
MGQVGRQLAIIGDDINR

SEQ ID NO: 241 - protein, 138 aa, Homo sapiens
MAKQPSDVSSECDREGRQLQPAERPPQLRPGAPTSLQTEPQDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 242 - protein, 135 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKADVVYNAFSLRV

SEQ ID NO: 243 - protein, 110 aa, Homo sapiens
MGAAMAGQEDPVQREIHQDWANREYIEIITSSIKKIADFLNSFDMSCRSRLATLNEKLTALERRIEYIEARVTKGETLTRTVPCCCWEVALHNTGHMGKAPAAFSSFLSP

SEQ ID NO: 244 - protein, 122 aa, Homo sapiens
MAAVLQQVLENAHIKLSNLYKSAADDSEAKSNELTRAVEELHKLLKEAGEANKAIQDHLLEVEQSKDQMEKEMLEKIGRLEKELENANDLLSATKRKGAILSEEELAAMSPTRGGINRGNIN

SEQ ID NO: 245 - protein, 19 aa, Homo sapiens
RWWMCGGRMEDMLCCRVGH

SEQ ID NO: 246 - DNA, 8 bp, Artificial Sequence
  Source Tag
aactcctc

SEQ ID NO: 247 - DNA, 8 bp, Artificial Sequence
  Source Tag
aatctacc

SEQ ID NO: 248 - DNA, 8 bp, Artificial Sequence
  Source Tag
aacaacac

SEQ ID NO: 249 - DNA, 8 bp, Artificial Sequence
  Source Tag
aatattcc

SEQ ID NO: 250 - DNA, 8 bp, Artificial Sequence
  Source Tag
ctcctaac

SEQ ID NO: 251 - DNA, 8 bp, Artificial Sequence
  Source Tag
ctttctct

SEQ ID NO: 252 - DNA, 8 bp, Artificial Sequence
  Source Tag
cttacttc

SEQ ID NO: 253 - DNA, 8 bp, Artificial Sequence
  Source Tag
atttcaat